(19) United States
(12) Patent Application Publication
(10) Pub. No.: US 2008/0132446 A1
(43) Pub. Date: Jun. 5, 2008

(54) Uses of neuronal pannexins for therapy and diagnosis in mammals

(75) Inventors: Hannah Monyer, Heidelberg (DE); Roberto Bruzzone, Paris (FR)

Correspondence Address: OBLON, SPIVAK, MCCLELLAND MAIER & NEUSTADT, P.C., 1940 DUKE STREET, ALEXANDRIA, VA, 22314, US

(73) Assignee: INSTITUT PASTEUR, Paris Cedex (FR)

(21) Appl. No.: 10/942,001

(22) Filed: Sep. 16, 2004

(30) Foreign Application Priority Data
Sep. 16, 2003 (EP) 03292281.7

(51) Int. Cl. A61K 38/17; C07K 14/47; G01N 33/53; A01K 67/027

(52) U.S. Cl. 514/12; 435/7.8; 800/9; 530/350

(57) ABSTRACT

The present invention is related to the use of neuronal pannexins for the manufacture of drugs for preventing and/or treating neurological disorders, particularly neurological disorders involving hippocampal pyramidal cells in mammals.

Moreover, the invention concerns the use of pannexins for in vitro diagnosing such neurological disorders.

The present invention is also directed to methods for selecting, in vitro or in vivo using an animal model, compounds useful for preventing and/or treating, in mammals, neurological disorders by modifying the channel-forming ability of pannexins.

The present invention relates to the industrial developments in connection with the domain of neurobiology. More precisely, the invention concerns medical and pharmaceutical applications of particular mammalian neuronal proteins.

The present invention is thus related to the use of pannexins, especially neuronal channel-forming pannexins, for the manufacture of drugs for preventing and/or treating neurological disorders, particularly neurological disorders involving hippocampal pyramidal cells in mammals.

Moreover, the invention concerns the use of pannexins for in vitro diagnosing such neurological disorders.

The present invention is also directed to methods for selecting, in vitro or in vivo using an animal model, compounds useful for preventing and/or treating, in mammals, neurological disorders, particularly neurological disorders involving the hippocampal pyramidal cells, by modifying the channel-forming ability of pannexins.

Gap junctions are collections of intercellular channels that, in vertebrates, are formed by connexins, a multi-gene family of which 20 members have been identified in humans (1). It is generally accepted that gap junctions between neurons represent the anatomical substrate of electrical synapses (reviewed in 2, 3). Although the incidence of electrical coupling relative to chemical synapses in the adult is relatively low, several studies have demonstrated that different types of interneurons of the hippocampus and neocortex communicate via electrical synapses in a cell-specific manner (4-11). Therefore, this additional form of intercellular communication appears to be more widespread than previously imagined and delineates independent networks of coupled cells. In this respect, electrical synapses have emerged as a common mechanism for synchronizing neuronal ensembles at different frequency bands, which have been proposed to underlie a variety of cognitive processes (e.g., perception, learning, and memory).

More precisely, besides the undisputed role of chemical transmission in network oscillations, both computer simulations and electrophysiological recordings have recently emphasized a key role for electrical synapses in the generation of synchronous activity in the hippocampus and neocortex (6, 12-17). Accordingly, the identification of connexin36 (Cx36) as the main neuronal connexin expressed in several areas of the brain (18) suggested that it may be an important component of gap junctions involved in the synchronization of large-scale neuronal networks. This possibility has been directly tested in mice with a targeted ablation of Cx36, which exhibit impaired electrical coupling in several brain regions (15, 19-23). Loss of this gap junction protein abolishes electrical coupling between hippocampal interneurons and disrupts gamma frequency network oscillations in vitro and in vivo (15, 24). The specificity of this impairment was indicated by the finding that high frequency rhythms in hippocampal pyramidal cells are unaffected by the lack of Cx36 (15).

These observations raise two possibilities: either a different connexin is specifically deployed throughout the pyramidal cell network or, alternatively, another class of molecules expressed in the mammalian brain forms electrical junctions between pyramidal cells. The latter hypothesis has received theoretical support from the discovery, in the database, of a novel family of genes encoding proteins for which the name “pannexins” has been proposed (25).

Pannexins are known to share structural features with gap junction proteins of invertebrates (innexins) and vertebrates (connexins) (25).

As shown for the first time in the context of the present invention, pannexins can form gap junction channels and, therefore, contribute to electrical communication in the nervous system of mammals. Indeed, it appears that pannexins form intercellular channels that allow communication between neurons, thus making a novel class of electrical synapses exhibiting some specific features compared to the channel-forming proteins characterized so far.

Interestingly, as shown herein, pannexins are the first proteins with gap junction-forming ability to be localized in the pyramidal cells of the hippocampus of mammals.

In contrast to connexins, the other class of proteins forming intercellular channels in mammals, which are mainly targeted to dendrites and form inter-dendritic electrical synapses, pannexins may also be axonally targeted and form axo-axonal gap junctions. This is the type of electrical synapses predicted to run between pyramidal cells.

On the basis of their cellular distribution in the brain (see the detailed description below), and their aforementioned specific features, pannexins are thought to underlie neurological disorders, including those involving hippocampal pyramidal neurons.

Therefore, thanks to their specific features, pannexins represent an advantageous tool for the development of drugs that will modify their channel forming properties.

Thus, the first aspect of the present invention concerns the use of at least one pannexin, especially one neuronal channel-forming pannexin, or at least one biologically active fragment thereof, or at least one biologically active derivative thereof, for the manufacture of a drug for preventing and/or treating, in a mammal, a neurological disorder, particularly a neurological disorder involving the hippocampal pyramidal cells.

The “use” as mentioned above encompasses any use of pannexins or biologically active fragments or derivatives thereof, for pharmaceutical purposes, which means the use of pannexins or biologically active fragments or derivatives thereof, either directly (e.g., the use of pannexins themselves as drugs), or indirectly. Examples of indirect uses of pannexins are the use as screening-tools for selecting compounds which may be used as drugs, or the use as starting materials for obtaining such compounds, via modification, transformation, etc . . . , of the pannexin structure.

As used herein, the term “pannexins” encompasses, but it is not limited to, neuronal channel-forming pannexins.

The terms and expressions <<gap junction proteins>>, <<channel-forming proteins>>, <<gap junction pannexins>>, <<channel-forming pannexins>> refer to the same proteins and can be used interchangeably.

For purposes of the invention, the terms “peptides”, “proteins” and “polypeptides” are synonymous. A “peptide” is a molecule comprised of a linear array of amino acid residues connected to each other in the linear array by peptide bonds. Such linear array may optionally be cyclic, i.e., the ends of the linear peptide or the side chains of amino acids within the peptide may be joined, e.g., by a chemical bond. Such peptides according to the invention may include from about three to about 500 amino acids, and may further include secondary, tertiary or quatemary structures, as well as intermolecular associations with other peptides or other non-peptide molecules. Such intermolecular associations may be through, without limitation, covalent bonding (e.g., through disulfide linkages), or through chelation, electrostatic interactions, hydrophobic interactions, hydrogen bonding, ion-dipole interactions, dipole-dipole interactions, or any combination of the above.

In addition, certain preferred peptides according to the invention comprise, consist essentially of, or consist of an allelic variant of pannexin. As used herein, an “allelic variant” is a peptide having from one to two amino acid substitutions from a parent peptide, but retaining the biological activity of interest of the parent peptide.

“Retaining the biological activity of interest of the parent peptide” means herein retaining the ability of pannexin to contribute to channel formation.

Peptides according to the invention can be conveniently synthesized using art recognized techniques.

Preferred peptidomimetics retain the biological activity of the parent peptide, as described above. As used herein, a “peptidomimetic” is an organic molecule that mimics some properties of peptides, preferably their biological activity, or interferes with said properties. Preferred peptidomimetics are obtained by structural modification of peptides according to the invention, preferably using unnatural amino acids, D amino acid instead of L aminoacid, conformational restraints, isosteric replacement, cyclization, or other modifications. Other preferred modifications include without limitation, those in which one or more amide bond is replaced by a non-amide bond, and/or one or more amino acid side chain is replaced by a different chemical moiety, or one of more of the N-terminus, the C-terminus or one or more side chain is protected by a protecting group, and/or double bonds and/or cyclization and/or stereospecificity is introduced into the amino chain to increase rigidity and/or binding affinity.

Still other preferred modifications include those intended to enhance resistance to enzymatic degradation, improvement in the bioavailability, and more generally in the pharmacokinetic properties, compared to a parent pannexin peptide.

All of these variations are well known in the art. Thus, given the peptide sequences of pannexin, those skilled in the art are enabled to design and produce peptidomimetics having binding characteristics similar to or superior to such peptides.

The peptides used in the therapeutic method according to the present invention may also be obtained using genetic engineering methods.

A person skilled in the art will refer to the general literature to determine which appropriate codons may be used to synthetize the desired peptide.

A method that allows a person skilled in the art to select in vitro, and optionally to purify a biologically active derivative that exhibits an agonist or an antagonist biological activity of a pannexin is described hereunder. According to this method, the selection of said biologically active derivative is performed via determining the changes induced by this candidate compound, such as the channel-forming ability involving a pannexin.

A biologically active derivative of a pannexin may be a protein, a peptide, a hormone, an antibody or a synthetic compound. A definition of the term “compound” is given hereafter.

In the context of the present invention, a “mammal” is an animal or a human. Preferably, by “mammal”, it is meant herein a human.

The present invention targets neurological disorders, i.e., disorders involving cells of the central nervous system (CNS), particularly brain cells and, more particularly, hippocampal pyramidal cells.

As used herein, the terms “neurological disorder, particularly neurological disorder involving the hippocampal pyramidal cells” mean any “disorder” (or “disease” or “trouble”) related to malfunctions of the cells, including impairments or disruptions of electrical coupling between cells, particularly between hippocampal pyramidal neurons. Since this electrical coupling is responsible for synchronizing neurons, said malfunctions or impairments or disruptions affect the following biological processes: ultra-fast oscillations in the hippocampus, memory storage, higher cognitive functions (e.g., perception, learning, memory), olfaction, and vision. Thus, a “neurological disorder involving the hippocampal pyramidal cells” as referred to herein is preferably selected from epilepsy, schizophrenia, memory disorders, Alzheimer's disease, pain disorders, visual deficits, visual acuity, odor discrimination, and olfaction deficits. For example, hippocampal sclerosis is a specific alteration of the hippocampus that is frequently observed in patients with temporal lobe epilepsy (69). In general, it is characterized by gliosis and neuronal loss, most prominently in the CA1 field of the hippocampus, followed by the hilus, CA4 and CA3 fields. In addition, this neuronal loss is accompanied by axonal reorganization involving both excitatory and inhibitory neurons.

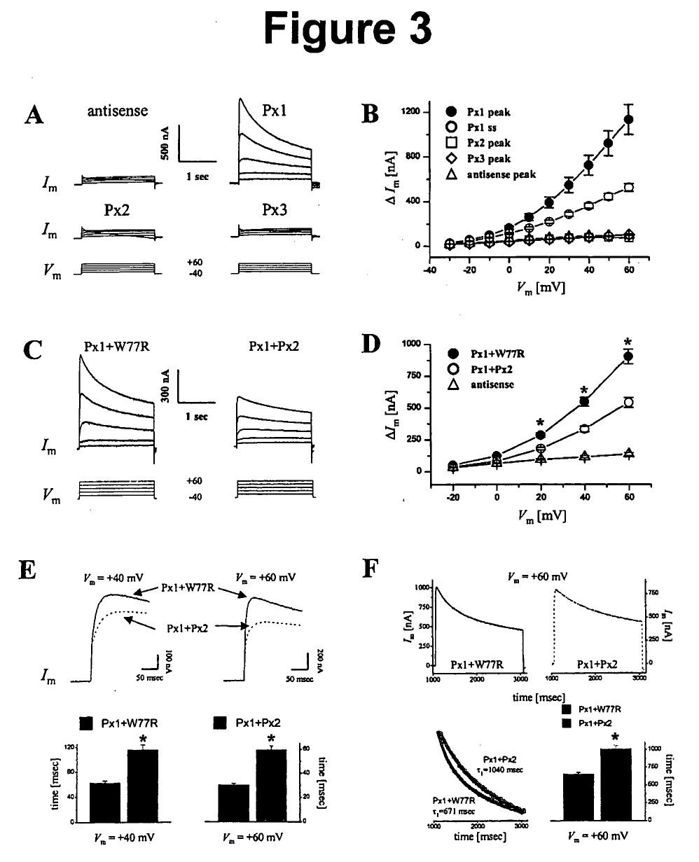

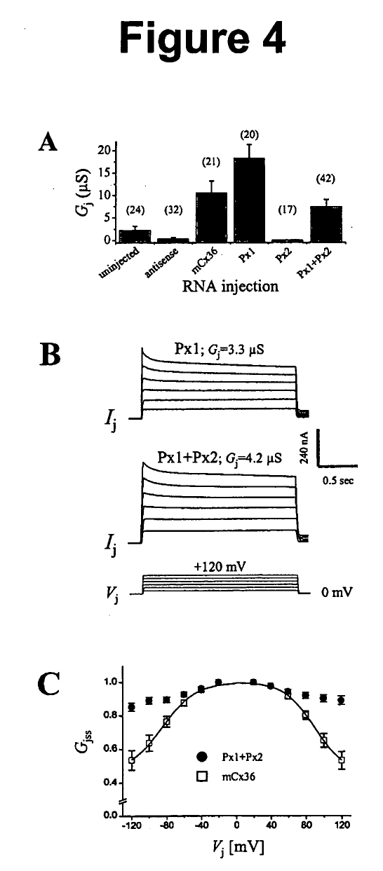

In a particular embodiment, the invention concerns the use of Pannexin2, or a biological active fragment thereof, or a biologically active derivative thereof, for the manufacture of a drug for preventing and/or treating, in a mammal, a neurological disorder, wherein modulation, preferably inhibition, of Pannexin1 is obtained. As shown below, Pannexin2, despite being unable to form functional channels by itself, reduces the amplitude of Pannexin1 currents, indicating that both proteins interact to form heteromeric channels with different properties (FIGS. 3 and 4). The interplay between these proteins implies that Pannexin2 is a modulator of Pannexin1 channel activity. Thus, a decrease in Pannexin2 expression or activity may result in an increased activity of Pannexin1 channels and an increased strength of coupling between neurons, whereas higher Pannexin2 levels or activity may depress channel activity.

The second aspect of the present invention is directed to the use of at least one neuronal pannexin for in vitro diagnosing, in a mammal, a neurological disorder, particularly a neurological disorder involving the hippocampal pyramidal cells.

Such a use for in vitro diagnostic entails advantageously at least one of the following:
mapping of a specific disorder to the chromosome region where the pannexin gene is located;
sequencing of said gene;
identification of at least one mutation comprised therein;
optionally testing the functional effects of said mutation;
establishing a genotype/phenotype correlation.

In a particular embodiment, said in vitro diagnostic comprises at least:
a) sequencing a pannexin gene in a mammal suspected to have a neurological disorder, particularly a neurological disorder involving the hippocampal pyramidal cells; and
b) identifying at least one mutation responsible for the lack of production of pannexin, or for the production of a pannexin the activity of which is modified compared to a control, for example the production of an inactive pannexin, in said mammal.

According to a third aspect, the present invention relates to methods for in vitro selecting a compound useful for preventing and/or treating, in a mammal, a neurological disorder, particularly a neurological disorder involving the hippocampal pyramidal cells. Advantageously, the selection is based upon the fact that said compound is capable of modifying the channel-forming ability involving a pannexin.

By “modifying or modulating the channel-forming ability of a pannexin” or “modifying or modulating the channel-forming ability involving a pannexin”, it is meant that said channel-forming ability is either induced (equivalents of “induced” being herein “increased”, “promoted”, “enhanced”, and “stimulated”), or inhibited (equivalents of “inhibited” being “reduced”, “decreased”, “suppressed”, and “blocked”). This may reflect, for instance, (i) an increase or decrease in expression or in activity of the pannexin polynucleotides or proteins; or (ii) a change in the amount of said polynucleotides or proteins, in the cellular distribution thereof, in the level of expression thereof, in the type of activity thereof.

In the context of the present invention, the term “polynucleotide” encompasses, but it is not limited to, RNA, DNA, RNA/DNA sequences of more than one nucleotide in either single chain or duplex form.

As used herein, the term “activity” when referring to a pannexin encompasses:
(i) the ability of said pannexin to constitute channels; and/or
(ii) when channels are formed, its ability to constitute functional channels.

In a first embodiment, such a method comprises at least:

a) measuring the channel-forming ability of, a pannexin in the absence of any compound (P0);
b) measuring the channel-forming ability of said pannexin in the presence of a compound (P1);
c) comparing P0 and P1; and
d) if P1 is significantly different from P0, selecting said compound.

On the one hand, In step d), if P1 is significantly greater than P0, the selected compound is an agonist of said pannexin.

By <<agonist>>, it is meant herein a compound capable of restoring or increasing the channel-forming ability and/or the amplitude and/or kinetics of the membrane currents involving a pannexin.

On the other hand, in step d), if P1 is significantly lower than P0, the selected compound is an antagonist of said pannexin.

An “antagonist” is herein a compound capable of inhibiting or decreasing the channel-forming ability and/or the amplitude and/or kinetics of the membrane currents involving a pannexin.

A “compound” herein refers to any type of molecule, biological or chemical, natural, recombinant or synthetic. For instance, such a compound may be a nucleic acid (e.g., an antisense or sense oligonucleotide including an antisense RNA), a peptide, a fatty acid, an antibody, a polysaccharide, a steroid, a purine, a pyrimidine, an organic molecule, a chemical moiety, and the like. Also encompassed by the term “compound” are fragments, derivatives, structural analogs or combinations of the above. In particular, the biologically active fragments or derivatives of pannexins, as defined above, are also encompassed by the term “compound”.

Methods for measuring channel-forming ability of proteins are well-characterized in the art. For instance, the skilled artisan, relying on the detailed description below, can perform the experimental procedure recited therein.

In a second embodiment, the method of the invention allows the selection of a compound of interest, based on its specific modulatory action on pannexins. In this respect, such a method for in vitro selecting a compound useful for preventing and/or treating, in a mammal, a neurological disorder, particularly a neurological disorder involving the hippocampal pyramidal cells, said compound being capable of specifically modifying the channel-forming ability of a pannexin, without modifying the channel-forming ability of a connexin, comprises at least:

a) measuring the channel-forming ability of each of said pannexin (P0) and said connexin (C0) in the absence of said compound;
b) measuring the channel-forming ability of each of said pannexin (P1) and said connexin (C1) in the presence of said compound;
c) comparing P0 and P1, and C0 and C1; and
d) if P1 is significantly different from P0, and if C1 is not significantly different from C0, selecting said compound.

As used herein, the expression “not significantly different” means “which does not appreciably modify the biological activity”. The term “significantly” is to be understood as being equivalent to “qualitatively significant”. In some particular embodiments, it may also encompass “quantitatively significant”.

Yet in this embodiment, the selected compound is either an agonist or an antagonist as defined above.

In a third embodiment, the method of the invention allows to select a compound of interest based on its ability to modulate the size of a channel formed by a pannexin. As an example of such a method, a control compound, or a bank of control compounds, the molecular size of which is known, is (are) used, this (these) compound(s) being advantageously labeled (e.g., by fluorescence) or naturally fluorescent. Such a method thus comprises at least the comparison of the movements, between a cell and the medium, of the control compound(s) in the presence and in the absence of the candidate compound. If a difference in the movements, between a cell and the medium, of the control compound(s) is observed, then the candidate compound is selected as being a compound of interest. Advantageously, this experiment is performed using a cluster of different control compounds having distinct molecular sizes in order to confirm the ability of the candidate compound to modulate the size of the channel.

A method of in vitro selecting a compound based on its ability to modulate the size of a channel formed by a pannexin, as illustred above, is also encompassed herein as a fourth aspect of the present invention.

In all embodiments described above, the method of selection advantageously further comprises purifying said selected compound.

Purification may be performed using standard techniques that are well known by the person skilled in the art.

By combining biochemical and electrophysiological approaches, it is here reported (see part B below) that pannexins exhibit a remarkable sensitivity to blockade by carbenoxolone (with an IC₅₀ of ˜5 μM), whereas flufenamic acid exerted only a modest inhibitory effect. The opposite was true in the case of connexin46 (Cx46), thus indicating that gap junction blockers are able to selectively modulate pannexin and connexin channels.

In this respect, gap junction blockers, such as carbenoxolone, may be useful as selective pannexin antagonists.

According to a fifth aspect, the present invention is directed to an animal model for in vivo selecting a compound useful for preventing and/or treating, in a mammal, a neurological disorder, particularly a neurological disorder involving the hippocampal pyramidal cells.

As indicated above, the selection is based upon the fact that said compound is capable of modifying the channel-forming ability involving a pannexin in said animal model.

In a particular embodiment, such an animal model is constructed by introducing at least one mutation in a pannexin gene of an animal, said mutation being responsible for the lack of production of pannexin, or for the production of a totally or partially inactive pannexin, said totally or partially inactive pannexin exhibiting a reduced or suppressed channel-forming ability, in said animal model.

In another embodiment, the present invention relates to an animal model, wherein a reporter gene is introduced into its genome, under the control of the endogenous promoter of a pannexin gene. Such an animal model allows to select a compound for its ability to modulate: (i) the expression of the protein encoded by said reporter gene; and/or (ii) channel formation.

According to a sixth aspect, the present invention concerns the use of aforementioned animal models for in vitro selecting a compound capable of modifying the channel-forming ability of a pannexin, said compound being useful for preventing and/or treating, in a mammal, a neurological disorder, including a disorder involving hippocampal pyramidal cells.

Thanks to the characterization of the properties of pannexin hemi-channel, it appears that they may account for movement, not only between cells of gap junction permeant molecules, even also across the non-junctional membrane, thus participating in additional brain functions:
the release of glutamate and/or other neurotransmitters:
the release of ATP and/or other nucleotides (such as cyclic ADP-ribose);
cellular death by apoptosis.

The functional impact of pannexin hemi-channels will be influenced by their ability to gate into the open configuration. The mechanisms that control the relative levels of unpaired and docked hemi-channels are not yet completely understood, although it had been previously suggested that, once two cells are paired, connexin hemi-channels tend to be progressively incorporated into gap junction channels. By contrast, data with paired oocytes do not suggest that this is the case with pannexins, since the amplitude of non-junctional currents did not decrease with time after pairing. Thus, it is more likely that both hemi-channels and intercellular channels coexist in cells where Pannexin 1 (see below) is expressed either alone or in combination with Pannexin 2 (see below). Pannexin hemi-channels will carry out paracrine and/or autocrine signals that may have physiological or deleterious consequences, depending on the metabolic conditions of the tissue. Pannexin hemi-channels may be altered in certain disorders and this illicit opening of hemi-channels will be pathogenetically relevant. Specifically, it appears that:
opening of pannexin hemi-channels is deleterious for cellular vulnerability to oxidative stress and ischemic injury;
opening of pannexin hemi-channels represents a means to convey long range signalling and contribute to molecular cross-communication between glial cells and neurons which could affect synaptic transmission and plasticity.

The present invention is illustrated, while not being limited, by the following figures:

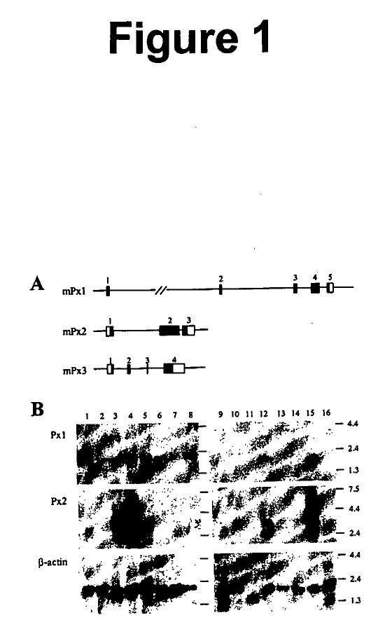

FIGS. 1A and 1B: Gene organization and mRNA expression in rodents.

FIG. 1A: The loci of the three pannexins (Px) in the mouse genome, indicating their exon (numbered boxed regions) and intron structure, are displayed. Within each exon, nucleotides contributing to the presumed protein sequence for each pannexin are shaded.

FIG. 1B: Northern blot analysis was performed on rat polyA+ RNA (lanes 1-16: adrenal gland, bladder, eye, spinal cord, thyroid, stomach, prostate, large intestine, testis, kidney, skeletal muscle, liver, lung, spleen, brain, heart). The Px1 probe hybridized to a 2.2-kb mRNA that was detectable in several organs including spinal cord and brain. The 3.5-kb Px2 was most abundant in spinal cord and brain and was also present in other organs. A less prominent 2.5-kb transcript was observed in some organs. Px3 mRNA was observed only in skin (not shown).

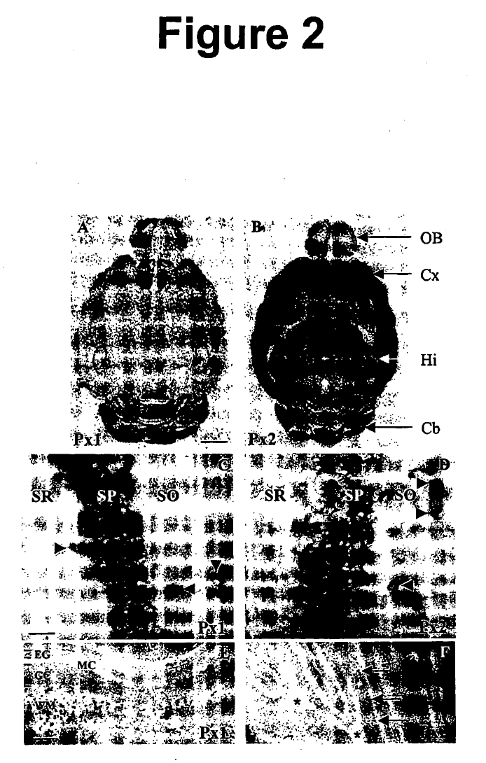

FIGS. 2A to 2F: Expression of Px1 and Px2 mRNA in the brain.

FIGS. 2A and 2B: The distribution of transcripts encoding Px1 and Px2 was determined by radioactive in situ hybridization in horizontal brain sections obtained from rats at postnatal day 15. X-ray autoradiograms illustrate a partially overlapping expression profile and indicate that they are abundant in the olfactory bulb (OB), cortex (Cx), hippocampus (Hi) and cerebellum (Cb). Scale bar is 2.5 mm.

FIGS. 2C to 2F: Non-radioactive in situ hybridization demonstrating that high expression of Px1 (FIG. 2C) and Px2 (FIG. 2D) was detected in the stratum pyramidalis (SP) of the hippocampus and in individual neurons (arrowheads) in the stratum oriens (SO) and stratum radiatum (SR). By contrast, in the cerebellum there was a strong labeling of Px1 expressing cells (FIG. 2E) in the white matter (WM) where Px2 expression was absent (FIG. 2F; asterisks). Note, however, that the Px2 riboprobe strongly labeled cells in the Purkinje cell layer (FIG. 2F; arrows). EG: external granule cell layer; MC: molecular cell layer; GC: granule cell layer. Scale bars are 50 μm (FIG. 2C-2D) and 250 μm (FIG. 2E-2F).

FIGS. 3A to 3F: Functional expression of pannexins in single Xenopus oocytes.

FIG. 3A: Whole-cell membrane currents (Im) were measured from single oocytes co-injected with pannexin RNAs and an oligonucleotide antisense to Xenopus Cx38 (see Materials and Methods). For clarity, representative traces are shown only in 20 mV increments.

FIG. 3B: Current-voltage relationships were determined for oocytes injected with either antisense oligonucleotides (open triangles), or Px1 (filled circles), Px2 (open squares), and Px3 (open diamonds) RNAs plus antisense. Peak current values above holding currents (ΔIm) were calculated and plotted as a function of Vm. Mean values from Px1-injected cells were significantly different (P<0.01) from those of control oocytes starting at a Vm of −10 mV. For Px1 steady-state currents (open circles), values recorded for 20 msec at the end of the pulse were averaged and plotted as above. Results are shown as mean ±SEM from at least 8 independent experiments. Antisense (n=45); Px1 (n=80); Px2 (n=46); Px3 (n=41).

FIGS. 3C to 3F: Functional interaction of Px1 and Px2 proteins. Antisense-treated oocytes were co-injected with Px1 RNA together with equal amounts of RNAs encoding either Px2 (dashed traces) or the W77R mutation of human Cx26 (black traces), which is devoid of functional activity (31).

FIGS. 3C-3D: Co-expression of Px1+Px2 reduced the amplitude of the outward currents induced by the depolarizing voltage steps (bottom traces). ΔIm recorded from Px1+Px2 (open circles) expressing oocytes was significantly less (*P<0.001) than that measured from Px1+W77R cells (filled circles). Results are shown as mean ±SEM from 4 independent experiments. Antisense (n=39); Px1+W77R (n=60); Px1+Px2 (n=67).

FIG. 3E: Px1+Px2 channels exhibit a delayed peak current time. Oocytes were depolarized to +40 mV (top left traces) and +60 mV (top right traces) from a holding potential of −40 mV. Peak currents were reached with a significant delay following the imposition of the voltage step (32 and 68 msec at +40 mV and 62 and 96 msec at +60 mV, for Px1+W77R and Px1+Px2, respectively). The lower panels show the mean SEM from 3 independent experiments for Px1+W77R (n=45) and Px1+Px2 (n=50); *P<0.001.

FIG. 3F: Px2 slows the kinetics of voltage-dependent closure of Px1 hemi-channels. Cells were depolanzed to +60 mV from a holding potential of −40 mV (top panels). Px1+Px2 hemi-channels (dashed line) gated more slowly than those formed by Px1+W77R (straight line). The tine-dependent decline in Im was well fit by a first order exponential decay function (lower left panel). The lower right panel illustrates the mean SEM from 3 independent experiments, for Px1+W77R (n=44) and Px1+Px2 (n=41); *P<0.001.

FIGS. 4A to 4C: Functional expression of pannexins in paired oocytes.

Cells were injected with the specified RNAs and manually paired in homotypic configuration (same construct in both oocytes).

FIG. 4A: Pairs of uninjected cells from the different batches of oocytes developed a variable level of junctional currents that exhibited the well-known voltage-dependent gating of endogenous Cx38 (42), whereas antisense controls showed negligible junctional conductance (Gj), indicating that endogenous currents had been suppressed. Oocyte pairs injected with either Px1 alone or in combination with Px2 (Px1+Px2) developed large junctional currents, whereas homotypic Px2-expressing pairs were uncoupled. Gj values recorded from oocytes expressing the neuronal mouse connexin36 (mCx36) are included for comparison. Results are shown as the mean ±SEM of the indicated number of oocyte pairs from 4-5 independent experiments.

FIG. 4B: Px1 and Px1+Px2 intercellular channels exhibit a weak sensitivity to transjunctional voltage (Vj). Junctional currents (Ij) were recorded from oocyte pairs in response to (Vj steps of opposite polarity (bottom traces) applied, from a holding potential of −40 mV, in 20 mV increments.

FIG. 4C: The plot shows the relationship of Vj to steady-state junctional conductance (Gjₛₛ), which was measured at the end of the Vj step and normalized to the values recorded at ±20 mV; Px1+Px2 (filled circles) and mCx36 (open squares). Data describing the Gj/Vj relationship were fit (smooth lines) to a Boltzmann equation, whose parameters were in agreement with those previously reported (43, 44). Results are shown as the mean ±SEM of 7-12 pairs (from 4 independent experiments) whose Gj was 3.2±0.8 μS and 4.8±1.1 μS for mCx36 and Px1+Px2, respectively. Because of the much larger non-junctional currents that were present in Px1 homotypic pairs, reliable Gjₛₛ/Vj plots with the complete polarization paradigm were difficult to obtain.

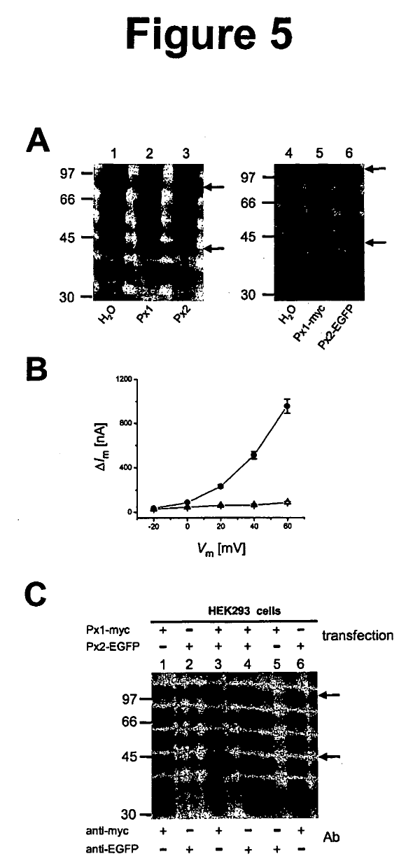

FIGS. 5A to 5C: Px1 and Px2 are expressed in heterologous systems and interact with each other.

FIG. 5A: The translational competence of RNAs encoding Px1, Px2 and the tagged constructs Px1-myc and Px2-EGFP was assessed in Xenopus oocytes. Cells injected with the specified RNAs exhibited specific polypeptide bands (arrows) that were easily discernible over the pattern of endogenous proteins (lanes 1 and 4) and migrated with an electrophoretic mobility similar to that of the in vitro synthesized products. The molecular mass (in kDa) and migration of protein standards are indicated on the left edge of each gel.

FIG. 5B: The current-voltage (I-V) relationship demonstrated that Px1-myc (filled circles) retained functional ability (n=22 cells). As expected, control oocytes (open triangles) showed no appreciable voltage-activated currents (n=4 cells). Peak current values above holding currents (ΔIm) from Px1-myc-injected cells were significantly different (P<0.01) from those of control oocytes starting at a membrane potential (Vm) of 20 mV. Results are shown as the mean ±SEM. When not visible, standard errors were comprised within the size of the symbol.

FIG. 5C: Co-immunoprecipitation of Px1 with Px2 expressed in HEK293. The antibodies (Ab) used are specified at the bottom of each lane. The molecular mass (in kDa) and migration of protein standards are indicated on the left edge of the gel. Px2-EGFP was pulled down with an anti-myc antibody only when co-transfected with Px1-myc and, conversely, Px1-myc was pulled down with an anti-EGFP antibody only when co-transfected with Px2-EGFP. Immunoprecipitation of Px1-myc yielded a doublet that may result from a partial degradation of the protein. Arrows point to the Px1-myc and Px2-EGFP protein bands. The lower intensity of the Px1-myc signal in single transfectants may have depended on a different transfection efficiency in this experiment and did not represent a consistent trend.

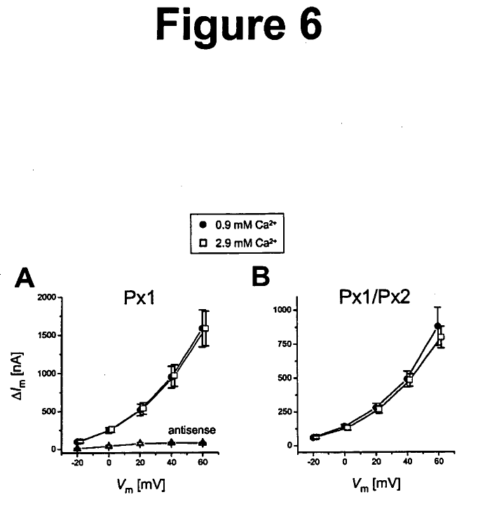

FIGS. 6A and 6B: Homomeric and heteromeric pannexin hemi-channels are not gated by extracellular Ca²⁺ concentrations.

The current-voltage (I-V) relationship of Px1 (FIG. 6A) and Px1/Px2 (FIG. 6B) hemi-channels obtained in control medium (filled circles) was not modified by incubating cells (5-15 min) in the presence of 2.9 mM Ca²⁺ (open squares). I-V plots obtained at 2.9 mM Ca²⁺ were slightly shifted to the right to allow a better visualization of the data points. Peak values above holding currents (ΔIm) are shown as the mean ±SEM of 20 (Px1) and 12 (Px1/Px2) oocytes. The I-V relationship of antisense-treated oocytes that were not injected with pannexin RNA (open triangles) is shown in A (n=4 cells). When not visible, standard errors were comprised within the size of the symbol. Vm, membrane potential.

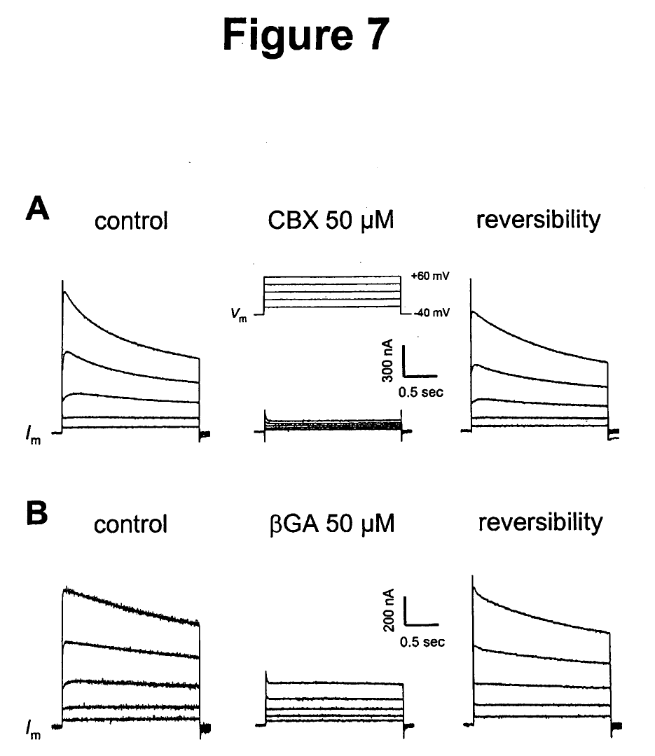

FIGS. 7A and 7B: The licorice derivatives carbenoxolone (CBX) and β-glycyrrhetinic acid (βGA) inhibit Px1 hemi-channel currents.

Top middle traces in FIG. 7A illustrate the experimental paradigm of depolarizing pulses (Vm, membrane potential). In control medium, expression of Px1 resulted in the activation of large outward currents when oocytes were stepped at positive potentials. Following a 30 min incubation with either CBX (FIG. 7A) or βGA (FIG. 7B) hemi-channel currents were strongly blocked, an effect that was reversible upon washout of the drugs and incubation (30 min) in control medium (reversibility). These traces are representative from a total of 8 (βGA) and 5 (CBX) cells.

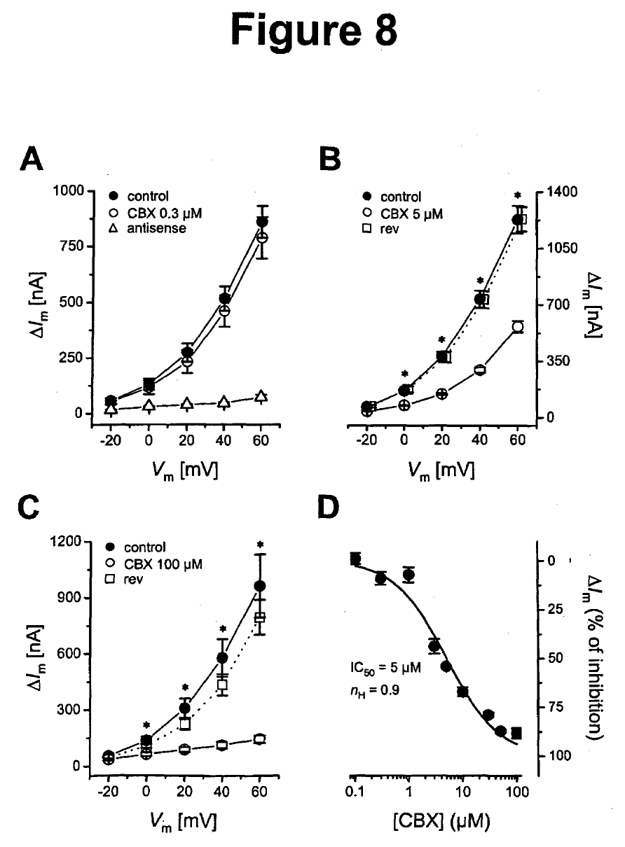

FIGS. 8A to 8D: Dose-dependent effect of carbenoxolone (CBX) on Px1 hemi-channel currents.

FIGS. 8A to 8C: Current-voltage (I-V) relationships were determined for oocytes that were first studied in control medium (filled circles) and then after a 15-30 min period in the presence of the specified CBX concentrations (open circles). Peak values above holding currents (ΔIm) were calculated and plotted as a function of membrane potential (Vm). The inhibitory action of CBX was dose-dependent and reversible upon washout of the drug (rev; open squares, dotted lines). Results are shown as the mean ±SEM of 4, 9 and 12 cells in FIGS. 8A, B and C, respectively. In FIG. 8B, the reversibility I-V curve was slightly shifted to the right to allow a better visualization of the data points. The I-V relationship of control oocytes that were not injected with Px1 RNA (antisense, open triangles) is shown in FIG. 8A (n=4 cells). *P<0.001 for control vs. CBX.

FIG. 8D: Semi-logarithmic plot illustrating the concentration dependence of the effect of CBX on Px1 hemi-channels. Each point represents the normalized peak currents (expressed as percentage of the values recorded in control medium) measured during the +60 mV depolarization step (mean ±SEM of 3-9 cells). The solid line is a fit of the data points to the Hill equation given in Microcal Origin 6.0 software. When not visible, standard errors were comprised within the size of the symbol.

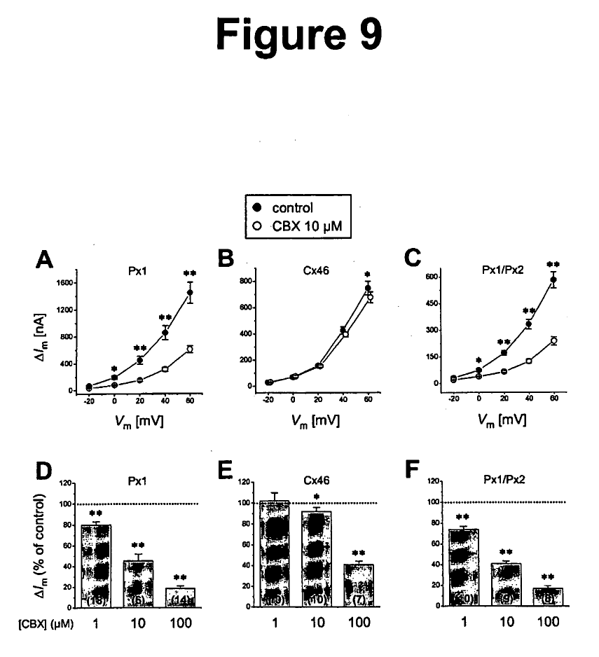

FIGS. 9A to 9F: Homomeric and heteromeric pannexin hemi-channels are more sensitive to carbenoxolone (CBX) than those formed by Cx46.

FIGS. 9A to 9C: Current-voltage (I-V) relationships were first recorded in control medium (filled circles) and then after a 15-30 min period in the presence of the 10 μM CBX (open circles). The inhibition of hemi-channel currents was reversible following washout of the drug (rev; open squares, dotted lines). Where necessary, I-V curves were slightly shifted to the right to allow a better visualization of the data points. Results are shown as the mean ±SEM of 6 (Px1), 10 (Cx46) and 9 (Px1/Px2) oocytes. *P<0.05; **P<0.005 for control vs. CBX.

FIGS. 9D to 9F: Comparison of the dose-dependent effect of CBX on pannexin and Cx46 hemi-channels. ΔIm measured during a +60 mV depolarization step in the presence of the specified CBX concentrations were normalized for each condition to the values obtained in control medium (dashed lines). Results are shown as the mean ±SEM of the number of cells indicated in parenthesis. *P=0.057; **P<0.005 vs. control values. When not visible, standard errors were comprised within the size of the symbol.

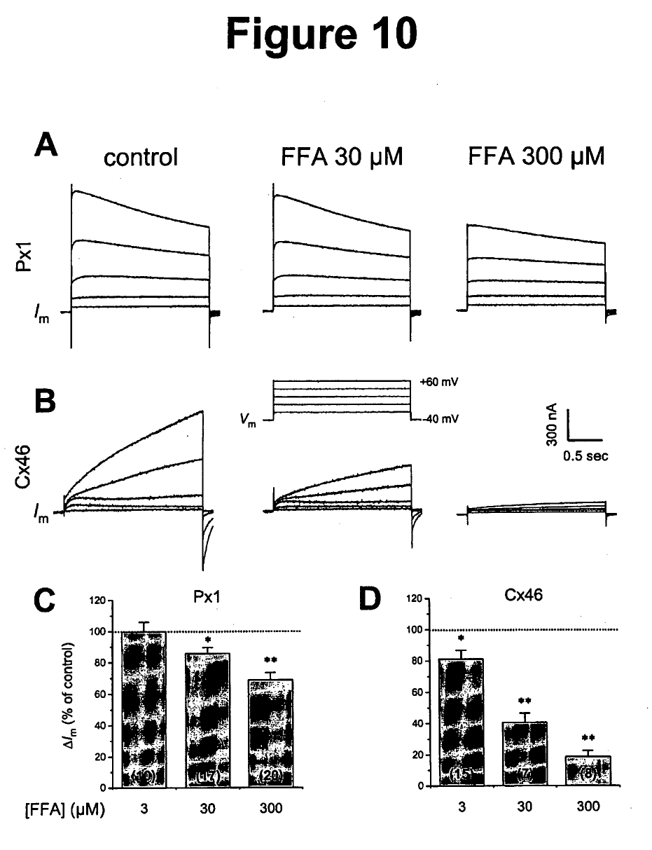

FIGS. 10A to 10D: Pannexin hemi-channels are less sensitive to flufenamic acid (FFA) than those formed by Cx46.

FIGS. 10A and 10B: Whole-cell membrane currents (Im) were measured from single oocytes expressing either Px1 or Cx46. Top middle traces in FIG. 10B illustrate the experimental paradigm of depolarizing pulses (Vm, membrane potential). The application of increasing concentrations of FFA induced a moderate inhibition of Px1 currents, in comparison to those recorded in control medium. By contrast, the same FFA concentrations virtually suppressed Cx46 hemi-channels. These traces are representative of the number of cells given in FIGS. 10C and 10D.

FIGS. 10C and 10D: Comparison of the dose-dependent effect of FFA on Px1 and Cx46 hemi-channels. Peak values above holding currents (DIm) measured during a +60 mV depolarization step in the presence of the specified FFA concentrations were normalized for each condition to the values obtained in control medium (dashed lines). Results are shown as the mean ±SEM of the number of cells indicated in parenthesis. *P<0.01; **P<0.001 vs. control values.

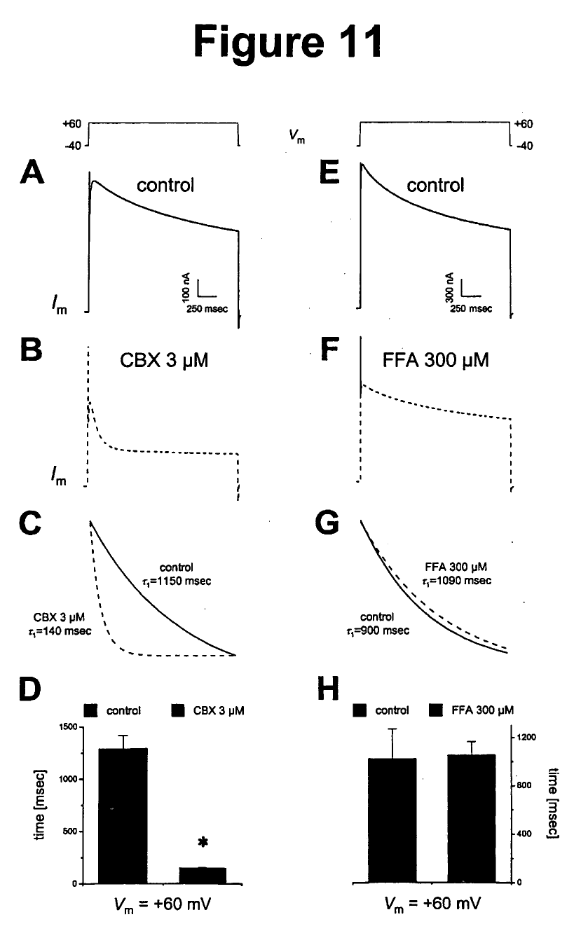

FIGS. 11A to 11H: Effects of carbenoxolone (CBX) and flufenamic acid (FFA) on the kinetics of voltage-dependent closure of Px1 hemi-channels.

Cells were depolarized to +60 mV from a holding potential of −40 mV (top traces, Vm).

FIGS. 11A and 11B: A representative current trace recorded in the presence of 3 μM CBX (dashed line) shows that Px1 hemi-channels gated much faster than in control medium (straight line).

FIGS. 11E and 11F: By contrast, 300 μM FFA (dashed line) did not affect the time constant of channel closure measured in control medium (straight line).

FIGS. 11C and 11G: A comparison of the time-dependent decline in Im (τ) is shown by superposing the re-scaled fits of the current traces shown above.

FIGS. 11D and 11H: The bar graphs show the mean ±SEM of 3 and 6 cells for CBX (FIG. 11D) and FFA (FIG. 11H), respectively. *P<0.01 for CBX vs. control values.

The present invention will be better understood in the light of the following detailed description of experiments, including examples. Nevertheless, the skilled artisan will appreciate that this detailed description is not limitative and that various modifications, substitutions, omissions, and changes may be made without departing from the scope of the invention.

### A. PANNEXINS FORM A NOVEL FAMILY OF GAP JUNCTION PROTEINS EXPRESSED IN BRAIN

### I. Experimental Procedures

### I-1-Molecular Cloning and mRNA Distribution.

cDNA clones were obtained by screening a rat hippocampal cDNA library (postnatal day 15) with [α-³²P] end labeled oligonucleotides complementary to nucleotides (nt) 181-225 and 316-360 of the mouse Px1 open reading frame (ORF), and to nt 199-243 and 334-378 of the human Px2 ORF (25). A probe for Px3 was generated by PCR using the oligonucleotide pair derived from nt 569-592 and 1059-1082 of the rat Px3 ORF, identified in the database. The tissue distribution of pannexin gene expression was investigated by reacting blots containing rat polyA+RNA (Rat MTN Blot I and II, catalog #77641 and 7795-1, respectively; Clontech, Palo Alto, Calif.) with [α-³²P] dCTP labeled probes derived either from the entire ORF of Px1 and Px2, or with a fragment derived from the β-actin transcript (supplied along with the blots).

For Northern blot analysis, the two filters were hybridized with probes for each of the three pannexins and exposed for 16 hrs at −70° C. (FIG. 1B).

Radioactive (26) and non-radioactive (27) in situ hybridization experiments were performed essentially as described previously.

[α-³⁵S] dATP end labeled oligonucleotides corresponded to nt 181-225 and 334-378 of the mouse Px1 and human Px2 coding sequence, whereas the entire rat ORF was used to generate digoxygenin-labeled sense and antisense riboprobes.

The distribution of transcripts encoding Px1 and Px2 was determined by radioactive in situ hybridization in horizontal brain sections obtained from rats at postnatal day 15 (FIG. 2, A-B).

### I-2-Functional Expression in Xenopus Oocytes.

The coding sequence of each pannexin was subcloned into the pBSxG expression vector (28). In vitro transcription, preparation of Xenopus oocytes, biochemical analysis and RNA injection were performed as described elsewhere (29). Metabolic labeling of oocytes indicated that all three pannexin RNAs directed the synthesis of specific polypeptide bands, whose electrophoretic mobility was similar to that of the in vitro translated constructs.

For physiological analysis, cells were injected with a total volume of 40 nl of either an antisense oligonucleotide (3 ng/cell) to suppress the endogenous Xenopus Cx38 (30), or a mixture of antisense plus the specified RNA (20-80 ng/cell). The ability of pannexins to form hemi-channels was assessed in single oocytes 2-4 days after RNA injection, using a two-electrode voltage-clamp.

Whole-cell membrane currents (Iₘ) were measured from single oocytes co-injected with pannexin RNAs and an oligonucleotide antisense to Xenopus Cx38 (FIG. 3A). Cells were initially clamped at a membrane potential (Vₘ) of −40 mV and depolarizing steps lasting 2 sec were applied in 10 mV increments up to +60 mV (bottom traces).

In FIG. 3B, peak current values above holding currents (ΔIm) were calculated and plotted as a function of Vₘ. Mean values from Px1-injected cells were significantly different (P<0.01) from those of control oocytes starting at a Vₘ of −10 mV. For Px1 steady-state currents (open circles), values recorded for 20 msec at the end of the pulse were averaged and plotted as above.

To investigate whether Px1 and Px2 could functionally interact, oocytes were co-injected with Px1 RNA (40-80 ng/cell) together with the specified amounts of RNAs encoding either Px2 or the W77R mutation of human Cx26, which is devoid of functional activity (31). To analyze whether pannexins formed intercellular channels, oocytes were stripped of the vitelline envelope 1-2 days after RNA injection, and manually paired in homotypic configuration (same construct in both oocytes) for 24-48 hr before measuring junctional conductance with a dual voltage clamp (FIG. 4). The setup, hardware and software used for electrophysiological measurements and data analysis were as previously described (29, 32). Both cells of a pair were initially clamped at −40 mV, and alternating pulses of ±10-20 mV were imposed to one cell (FIG. 4A). The current delivered to the cell clamped at −40 mV during the voltage pulse is equal in magnitude to the junctional current and can be divided by the voltage to yield the value of junctional conductance (Gⱼ).

In FIG. 4B, junctional currents (Iⱼ) were recorded from oocyte pairs in response to Vⱼ steps of opposite polarity (bottom traces) applied, from a holding potential of −40 mV, in 20 mV increments.

In FIG. 4C, the steady-state junctional conductance Gⱼₛₛ was measured at the end of the Vⱼ step and normalized to the values recorded at ±20 mV; Px1+Px2 (filled circles) and mCx36 (open squares). Data describing the Gⱼ/Vⱼ relationship were fit (smooth cyanide lines) to a Boltzmann equation, whose parameters were in agreement with those previously reported (43, 44).

### I-3-Statistical Analysis.

Results are shown as mean ±SEM. An independent experiment is defined as a series of data obtained with oocytes isolated from one animal. Comparisons between two populations of data were made using the Student's unpaired t-test. P values of 0.01 or less were considered to be significant.

### I-4-Database Accession Numbers.

The rat pannexin cDNAs were assigned the following accession numbers: AJ557015 (Px1); AJ557016 (Px2); AJ557017 (Px3).

The human pannexins Px1, Px2, and Px3 were assigned accession numbers NP_{—}056183, NP_{—}443071, and NP_{—}443191, respectively. The mouse pannexins Px1, Px2, and Px3 were assigned accession numbers NM_{—}019482, NM_{—}001002005 and NM_{—}172454, respectively.

### II. Results

### II-1-Structure and Organization of the Pannexin Genes.

Analysis of the cDNA sequences for Px1, Px2 and Px3 identified open reading frames (ORFs) encoding proteins with calculated molecular mass of 48,072, 73,270 and 44,976 daltons, respectively. The sequences of all three proteins predict, like for connexins, four transmembrane domains and cytoplasmic amino- and carboxy termini. A hallmark of gap junction-forming proteins is the presence of conserved, regularly spaced cysteine residues located on the two extracellular loops. Whereas the connexins contain three such residues, pannexins contain only two, thus resembling in this respect innexins, the invertebrate constituents of intercellular channels (33). A comparison of the cDNAs to the mouse genomic sequence (obtained from the Ensembl database; http://www.ensembl.org) resulted in the determination of the exon-intron structure of the three mouse pannexin genes (FIG. 1A). Considerable variability was found in the organization and length of the three gene loci, the protein coding regions could be assigned to five, three, and four exons respectively, for the Px1, Px2, and Px3 genes.

### II-2-Distribution of Pannexin mRNA.

Northern blots indicated that Px1 and Px2 transcripts were co-expressed in many tissues including eye, thyroid, prostate, kidney, liver and CNS (FIG. 1B). Px1 probe hybridized to a 2.2-kb mRNA that was detectable in several organs including spinal cord and brain (FIG. 1B). The 3.5-kb Px2 was most abundant in spinal cord and brain and was also present in other organs (FIG. 1B). A less prominent 2.5-kb transcript was observed in some organs. By contrast, Px3 transcripts could only be detected in the skin, which was found, by RT-PCR, to be devoid of Px1 and Px2 mRNA. In situ hybridization studies demonstrated widespread expression of both transcripts in many brain regions, including cortex, striatum, olfactory bulb, hippocampus, thalamus, cerebellum. FIG. 2, A and B, illustrate a partially overlapping expression profile and indicate that the transcripts encoding Px1 and Px2 are abundant in the olfactory bulb, cortex, hippocampus, and cerebellum. No signal was detected in parallel competition experiments with an excess of unlabeled probe.

Upon closer inspection at the cellular level, a differential distribution of Px1 and Px2 mRNA was apparent. In hippocampus, for example, both Px1 (FIG. 2C) and Px2 (FIG. 2D) were expressed in the pyramidal cell layer and in individual neurons (arrowheads) in the stratum oriens and stratum radiatum. Based on their location, the scattered neurons (NeuN positive) can be inferred to be GABAergic interneurons. By contrast, in the cerebellum, Px1 expressing cells (FIG. 2E) were abundant in the white matter where Px2 expression was absent (FIG. 2F, asteriks; note, however, the high Px2 labeling in the Purkinje cell layer in FIG. 2F, arrows). The labeling of Px1 expressing cells in the white matter was not restricted to the cerebellum but was also observed in other white matter structures (e.g. corpus callosum, fimbria formix), which, similarly, were also devoid of Px2 expression.

No staining was obtained with sense probes.

### II-3-Functional Expression in Single Xenopus Oocytes.

Large, voltage-activated outward currents were consistently induced when oocytes expressing Px1 were stepped to voltages greater than −20 mV (FIG. 3A-B). At large positive potentials, Px1 currents reached a peak within 30-60 msec of the imposition of the voltage step and then declined slowly, this rectification becoming more pronounced with increasing positive potentials. By contrast, neither Px2 nor Px3 induced membrane currents above those recorded in controls (FIG. 3A-B). Furthermore, incubation of oocytes for 10-30 min in carbenoxolone completely suppressed Px1 currents (peak amplitudes at +60 mV were 1189±170 and 255±47 nA for control medium and 30 μM carbenoxolone, respectively; n=4) and this effect was fully reversible (peak amplitude at +60 mV after a 30 min recovery period in control medium was 963±239 nA; n=4). Since the in situ hybridization studies revealed co-expression of Px1 and Px2, subsequent investigations entailed a more detailed functional analysis of these two proteins. To test whether they could form heteromeric channels, currents were recorded from oocytes co-expressing Px1 with Px2 (40-80 ng of RNA/cell) and were found to be significantly reduced with respect to those measured from cells that had been injected with the same amount of Px1 RNA. To exclude the possibility that this behavior was merely due to overloading of the synthetic machinery given the difference in the total amount of RNA injected, oocytes were injected with equal amounts of RNA for Px1 and the W77R mutation of human Cx26 (40-80 ng each/cell), which is devoid of functional activity (31). These experiments showed a reduction in current amplitude, suggesting that Px1+Px2 channels that are different from those composed of Px1 alone (FIG. 3C-D). As illustrated, ΔIₘ recorded from Px1+Px2 expressing oocytes was significantly less (*P<0.001) than that measured from Px1+W77R cells.

Given that Px2 expression in the brain appears to be much stronger than Px1, whether Px2 could simply function as a dominant negative partner was also tested by co-injecting Px1 and Px2 RNAs at a 1:5 ratio (40:200 ng/cell for Px1:Px2, respectively). Current amplitudes recorded at +60 mV were similar, irrespective of whether Px1 and Px2 were injected at a ratio of 1:1 (591±40 nA; n=27) or of 1:5 (517±45 nA; n=20), further indicating that they both interact and form functionally heteromeric channels. Interestingly, a decrease in current amplitude was not observed when voltage-activated currents were measured from oocytes receiving RNAs for Px1 and Px3, which are not co-expressed in rat tissues. Moreover, following the imposition of a voltage step, Px1+Px2 channels reached peak currents with a significant delay compared to Px1 expressing cells (FIG. 3E), which could result from slower opening or slower closing or both. In FIG. 3E, oocytes were depolarized to +40 mV (top left traces) and +60 mV (top right traces) from a holding potential of −40 mV. Peak currents were reached with a significant delay following the imposition of the voltage step (32 and 68 msec at +40 mV and 62 and 96 msec at +60 mV, for Px1+W77R and Px1+Px2, respectively).

Finally, as illustrated in FIG. 3F, analysis of the kinetics of channel closure at the more positive membrane potentials, revealed that currents recorded from Px1+Px2 expressing cells, presumably reflecting heteromeric hemi-channels, gated more slowly than those measured from oocytes injected with Px1+W77R RNA, presumably reflecting homomeric Px1 hemi-channels. Px2 slows the kinetics of voltage-dependent closure of Px1 hemi-channels. Cells were depolarized to +60 mV from a holding potential of −40 mV (top panels). The time-dependent decline in Iₘ was well fit by a first order exponential decay function (lower left panel; cyanide line superposed to the re-scaled current traces shown above).

### II-4-Functional Expression in Paired Xenopus Oocytes.

Px1 alone and in combination with Px2 induced the assembly of intercellular channels, whereas Px2 alone failed to do so (FIG. 4A). As illustrated, pairs of uninjected cells from the different batches of oocytes developed a variable level of junctional currents that exhibited the well-known voltage-dependent gating of endogenous Cx38 (42), whereas antisense controls showed negligible coupling, indicating that endogenous currents had been suppressed. It should be noted that intercellular channels were consistently detected only from batches of oocytes in which a robust junctional conductance was recorded with homotypic pairs expressing either mouse Cx36 (FIG. 4A) or human Cx26 wild-type, which served as positive controls. In this series of experiments, 20 out of 23 Px1 pairs and 36 out of 42 Px1+Px2 pairs were coupled. As shown in FIG. 4B, both Px1 and Px1+Px2 pairs displayed a remarkable insensitivity to transjunctional potentials of opposite polarities (Vⱼ). Thus, with a driving force ≦±60 mV, junctional currents varied linearly with voltage (FIG. 4B) whereas, at higher transjunctional potentials, the conductance of Px1+Px2 channels displayed only a very modest reduction (˜15%) of the initial values (FIG. 4C), similar to what reported for crayfish septate junctions (34) and human Cx31.9 (32). In FIG. 4C, the plot shows the relationship of Vj to steady-state junctional conductance (Gⱼₛₛ). Because of the much larger non-junctional currents that were present in Px1 homotypic pairs, reliable Gⱼₛₛ/Vⱼ plots with the complete polarization paradigm were difficult to obtain.

Although it has been reported that junctional currents measured in insect cells are sensitive to changes in membrane potential (35), the relative voltage insensitivity of pannexin intercellular channels with polarization of one cell is a strong indication that polarization of both cells is not likely to affect significantly junctional conductance.

### B. PHARMACOLOGICAL PROPERTIES OF HOMOMERIC AND HETEROMERIC PANNEXIN HEMI-CHANNELS EXPRESSED IN XENOPUS OOCYTES

### I. Materials and Methods

### I-1-Molecular Cloning, In Vitro Transcription and Translation

The carboxyl-terminally modified pannexin constructs were prepared in the pRK5 expression vector by introducing either an epitope tag [from either c-myc or the influenza virus hemagglutinin (HA) genes], or the entire enhanced Green Fluorescent Protein (EGFP) coding portion fused in frame with the rat pannexin sequence in which the stop codon had been mutated. All constructs were sequenced to verify that PCR reactions had not introduced unwanted mutations. For functional expression studies in Xenopus oocytes, Px1, Px2 and the respective tagged constructs were subcloned into the pBSxG expression vector, as described previously. All constructs were linearized with Xho I (MBI Fermentas, St. Leon-Roth, Germany) and capped RNAs were transcribed in vitro with T7 RNA polymerase using the mMessage mMachine kit (Ambion, Austin, Tex.) according to the manufacturer's instructions. The purity and concentration of different RNA batches were assessed by measuring absorbance at 260/280 nm. The translational competence of each RNA was tested using a rabbit reticulocyte lysate system (Promega, Madison, Wis.) in the presence of ³⁵S-methionine (Amersham Europe, Otelfingen, Switzerland), as detailed elsewhere (49). Radioactive products were separated on a 10% sodium dodecyl sulphate (SDS)-polyacrylamide gel and visualized by fluorography (X-Omat AR film; Eastman Kodak, Rochester, N.Y.). As expected, synthetic RNAs directed the synthesis of specific polypeptide bands, whose electrophoretic mobility was consistent with their deduced molecular mass. The expression vectors for rat Cx46 (37) and zebrafish Cx52.6 (50) have been previously described.

### I-2-Preparation, Microinjection and Metabolic Labeling of Xenopus Oocytes

Adult Xenopus laevis females, purchased from Nasco (Fort Atkinson, Wis.), were anesthesized according to the approved protocols of the Central Animal Facility of the University of Heidelberg, and approximately ⅔ of one ovarian lobe was carefully excised. Animals were allowed to recover from surgery and were used not more than three times a year. Isolation of Xenopus oocytes, biochemical analysis and RNA injection were performed as previously described (49). Briefly, for metabolic labeling the RNA-injected oocytes (80-100 ng/cell) were incubated at 18° C. for 12-20 hours in Modified Barth's medium (hereafter referred to as “control medium”) (51) supplemented with 3S-methionine (0.5 μCi/μl). Cells homogenates were dried, resuspended in sample buffer (25 mM Tris-HCI pH 6.8, 4% SDS, 10% glycerol, 0.01% bromophenol blue, 2% β-mercaptoethanol), electrophoresed ( 1/10 of an oocyte/lane) on a 10% SDSpolyacrylamide gel and analyzed by fluorography (overnight exposure) as described above.

### I-3-Cell Culture and Immunoprecipitation

Human embryonic kidney (HEK) 293 cells were cultured in Dulbecco's Modified Eagle Medium (DMEM; Invitrogen, Carlsbad, Calif.), supplemented with 5% heatinactivated fetal calf serum (FCS), according to standard protocols. The choice of a cell line for this series of experiments was based on previous observations that overexpressed connexins have a tendency to aggregate in unspecific fashion in cell homogenates of Xenopus oocytes (52), thereby complicating the interpretation of immuneprecipitation data. HEK293 were transfected at a 60% confluency with 3 μg each of Px1-myc and Px2-EGFP (both in pRK5), either separately or in combination, using the calcium phosphate method. Cells were cultured for 3 days, up to −90% confluency, before biochemical analysis was performed and the efficiency of transfection (around 70%) was routinely checked by evaluating the proportion of positive cells for Px2-EGFP with a fluorescence microscope. For immuneprecipitation experiments, cells were starved for methionine during 30 min at 37 C in “labeling medium” (DMEM lacking methionine, supplemented with 5% FCS and 2 mM glutamine) under ambient CO₂ conditions in a tissue culture incubator (52). Cells were washed and incubated for 20 min at 37° C. with fresh labeling medium (2.5 ml/35-mm dish) containing [³⁵S]-methionine (0.1 mCi/ml). Dishes were chilled on ice for 2 min and then placed in the same low CO₂ incubator for 4 h at 20° C. At the end of the labeling period, cells were rinsed three times with immuneprecipitation (IP) buffer (138.8 mM NaCl, 5.36 mM KCl, 0.336 mM Na₂HPO₄, 0.345 mM KH₂PO₄, 0.8 mM MgSO₄, 2.7 mM CaCl₂, 20 mM HEPES, pH 7.5) supplemented with 10 mM NEM (N-ethyl-maleimide; Sigma-Aldrich, Steinheim, Germany) and Complete® protease inhibitor (Roche Diagnostics, Mannheim, Germany). Cells were scraped in IP buffer, the dish was washed with IP and the cell suspension was centrifuged at 180×g in a table top centrifuge for 10 min at 4° C. The cell pellet was resuspended in IP buffer (one confluent 60-mm dish/1 ml) and homogenized by repeated aspiration through a 25-gauge needle. Following the addition of Triton X-100 (1% final concentration), the homogenate was incubated on ice for 30-40 min and eventually centrifuged at 100,000×g in a tabletop Beckman (TL-100) ultra-centrifuge 60 min at 4° C. The Triton-soluble supematant was collected and aliquots (⅕ of a 35-mm dish) were then incubated overnight at 4° C. on a rotating plate in the presence of 4 μg of the desired antibody: either a mouse monoclonal anti-myc (catalog #sc-40; Santa Cruz Biotechnology, Santa Cruz, Calif.) or a mouse monoclonal anti-EGFP antibody (catalog #AFP5002; Quantum Biotechnologies, Montreal, Canada). To precipitate immune complexes, 25 μl of Protein A-Agarose slur (Santa Cruz Biotechnology) were added for 1 h at 4° C. under rotation. Samples were centrifuged in a refrigerated tabletop centrifuge for 1 min at 13,000 rpm, the supematant was discarded and Protein A-Agarose beads were washed 4× with cold buffer, containing: 100 mM NaCl, 20 mM sodium borate, 15 mM EDTA, 15 mM EGTA, 0.02% NaN₃, pH 8.5. During the first three washing steps, the buffer was supplemented with 0.5% bovine serum albumin (BSA), 0.5% Triton X-100, 0.1% SDS, 10 mM NEM and Complete® protease inhibitor, whereas in the last wash BSA was omitted and the Triton X-100 concentration was reduced to 0.05. The final pellet was solubilized in 30 μl electrophoresis sample buffer and boiled for 5 minutes. Aliquots (10 μl) were loaded onto a 10% SDS-polyacrylamide gel and visualized by fluorography (X-Omat AR film, two weeks exposure), as described above.

### I-4-Electrophysiology and Pharmacology

Stage V-VI cells were individually selected under a dissecting microscope and cultured thereafter in control medium at 18° C. For physiological analysis, all cells were injected with a total volume of 40 nl of either an antisense oligonucleotide (3 ng/cell) to suppress the endogenous Xenopus Cx38 (30; 51), or a mixture of antisense (as above) plus the specified pannexin or connexin RNA. The following amounts of RNAs were injected: 40-80 ng/cell for Px1 and Px2 RNAs, either separately or co-injected at a 1:1 ratio; 4 ng/cell for Cx46; 10-20 ng/cell for Cx52.6. For analysis of Cx46 hemi-channels, the extracellular Ca²⁺ concentration was raised to 2.9 mM to prevent the lysis of the injected oocytes that occurs in control medium, which contains 0.9 mM Ca²⁺ (53). To characterize hemi-channel activity, current recordings were performed in single oocytes 24-96 hr after RNA injection, using a two-electrode voltage-clamp procedure.

The setup, hardware and software used for electrophysiological measurements and data analysis were as previously described (54; 49). Cells were clamped at −40 mV, and whole cell currents recorded in response to depolarizing voltage steps (from −20 to +60 mV in 20 mV increments) imposed for 2 sec. Current outputs were filtered at 200 Hz and sampled at 500 Hz.

All experiments were carried out at 18° C. Carbenoxolone (the succinyl ester of glycyrrhetinic acid), β-glycyrrhetinic acid and flufenamic (Sigma-Aldrich) were prepared freshly by dissolving them in either water (in the case of carbenoxolone) or dimethyl sulfoxide (DMSO), whose final concentration never exceeded 0.1%. Under these conditions, DMSO did not affect the characteristics of the currents recorded from either RNA-injected or control cells. Oocytes were placed on a Teflon tubing in a perfusion chamber and drugs were delivered using an electronically controlled gravity flow perfusion system (ALA Scientific Instruments, Westbury, N.Y.). Cells were incubated for 15-30 minutes in the presence of the different drugs and reversibility was recorded after extensive washings in control medium. Current-voltage (I-V) relationships were generated by plotting peak current values above holding currents (ΔIm) versus membrane potential. The time constants (τ) of voltage-dependent transitions of hemi-channel currents were calculated using data fitting functions in Origin 6.0 (Microcal Software, Northampton, Mass.). In all experiments, positive controls showing the effect of the tested drugs were routinely included. To exclude that the experimental results (see below) were influenced by the different external Ca²⁺ concentrations in which cells expressing pannexins and Cx46 were incubated (0.9 and 2.9 mM, respectively), in some experiments the effect of the same drugs on pannexin hemi-channels was tested in 2.9 mM Ca²⁺, which per se does not affect the amplitude of pannexin currents (see FIG. 6). The pharmacological behavior of pannexins in high external Ca²⁺ was unchanged and, therefore, these data were pooled with those obtained in control medium.

### I-5-Statistical Analysis

Results are presented as means ±SEM of the specified number of cells. Data were pooled from a minimum of two independent experiments (viz., oocytes isolated from different animals). Comparisons between two populations of data were made using the Student's paired t-test with a confidence limit for significance set at 0.05 or less.

### II. Results

### II-1-Biochemical Evidence for Interaction of Px1 with Px2

Evidence that Px2 could not assemble homomeric channels, but reduced the amplitude and modified the voltage gating kinetics of Px1 hemi-channels was previously obtained, as described above, suggesting that heteromeric Px1/Px2 channels were formed. To obtain a direct biochemical evidence for an interaction between the two pannexins, Px1-myc and Px2-EGFP tagged constructs were prepared and their translational competence in metabolically labeled Xenopus oocytes was checked. Based on the presence of specific bands of the expected molecular mass, it was concluded that the two tagged pannexins were efficiently synthesized in a heterologous expression system (FIG. 5A). To verify that the myc-tag did not alter the functional ability of Px1, it was next determined the current-voltage (I-V) relationship of single oocytes injected with synthetic RNA encoding Px1-myc. Stepwise depolarization of oocytes expressing Px1-myc resulted in the appearance of large, voltage activated outward currents that were similar in amplitude and kinetics to those recorded with Px1, indicating that the essential properties of Px1 hemi-channels were not modified by the addition of a myc-tag (FIG. 5B). To examine whether Px1 and Px2 can interact, HEK293 cells were transfected with two differently tagged constructs, Px1-myc and Px2-EGFP. When the two tagged pannexins were co-transfected, the anti-myc antibody pulled down an additional band whose migration was undistinguishable from that of Px2-EGFP (FIG. 5C, cf. lanes 2 and 3). Similarly, in the reciprocal experiment, the anti-EGFP antibody pulled down a band that exhibited the same mobility as Px1-myc (FIG. 5C, cf. lanes 1 and 4). Control experiments showed that no bands were detected when either the anti-EGFP antibody was added to lysates of cells transfected only with Px1-myc or the anti-myc was used to precipitate Px2-EGFP transfectants (FIG. 5C, lanes 5-6, respectively). Similar results were also obtained by co-transfecting Px1-myc with an influenza virus hemagglutinin (HA) epitope tag fused at the carboxylterminus of Px2, Px2-HA, using the appropriate antibodies for immuneprecipitation. Together, these findings support a specific interaction between Px1 and Px2.

### II-2-Homomeric and Heteromeric Pannexin Hemi-Channels are not Gated by External Ca²⁺

Since previous studies have demonstrated that the activation of several connexion hemi-channels is critically dependent on the concentration of divalent cations in the culture medium (53; 55; 56; 57; 58; 59; 50), whether external Ca²⁺ could modulate homomeric Px1 and heteromeric Px1/Px2 hemi-channel conductance was analyzed. As previously reported, heteromeric Px1/Px2 hemi-channels exhibited reduced current amplitudes and modified voltage gating kinetics with respect to homomeric Px1 analyzed in the same batch of oocytes. Plots of the current-voltage (I-V) relationship indicated that, when the extracellular Ca²⁺ concentration was raised from 0.9 mM (the concentration present in control medium) to 2.9 mM, the peak current amplitude of Px1 hemi-hannels was totally unaffected (FIG. 6A). Moreover, the macroscopic levels of Px1 hemi-channel currents recorded at +60 mV remained unchanged even in the presence of 10 mM extracellular Ca²⁺ (1525±187 nA in control medium vs. 1749±180 nA in 10 mM Ca²⁺; n=11 cells), or in nominally Ca²⁺-free solution (1760±247 in control medium vs. 1796±208 in nominally Ca²⁺-free solution; n=14 cells). Similarly, the current amplitudes of Px1/Px2 hemi-channels measured in 2.9 mM Ca²⁺ were virtually identical to those recorded in control medium (FIG. 6B). In contrast, hemichannel currents of zebrafish Cx52.6, which has been recently shown to make Ca²⁺-sensitive hemi-channels and, therefore, was used as a control in this series of experiments, were drastically inhibited at the +60 mV depolarization step by raising the external Ca²⁺ concentration (from 1278±212 nA in control medium to 632±120 nA in 2.9 mM Ca²⁺; n=11 cells).

### II-3-Licorice Derivatives are Potent Blockers of Pannexin Hemi-Channels

Several compounds derived from licorice root have been widely used over the past decade as pharmacological tools to block connexin channels (60; 61). Thus, it was chosen to investigate the effects of two of these molecules, the β-stereoisomer of 18-glycyrrhetinic acid (βGA) and its synthetic derivative carbenoxolone (CBX), on Px1 hemi-channels (FIG. 7). Incubation of oocytes in the presence of 50 μM of either CBX (FIG. 7A) or βGA (FIG. 7B), a concentration within the range of those used to maximally inhibit gap junction channels, resulted in a robust and reversible decrease of Px1 hemi-channel currents.

Since CBX appeared to cause a stronger blockade than βGA and is considered to be devoid of major side effects that plague other commonly used connexin inhibitors (62; but see also 63), further analysis was carried out only with this drug. It was observed that very low CBX concentrations (<1 μM) were ineffective, whereas a dose-dependent inhibition over the entire I-V relationship occurred at higher concentrations (FIG. 8A-C). These effects were already detectable following a 5-15 min incubation in CBX-containing medium, did not appear to change significantly over time (up to 30-60 min) and were always reversible, even at the highest dose that was tested (100 μM). Thus, after washout of the drug and a further 30 min incubation in control medium, the amplitude of voltage-activated Px1 hemi-channels was restored to almost the same levels measured before application of CBX (FIGS. 8B-C). The concentration dependence of CBX-induced blockade of Px1 hemi-channel currents was determined by exposing 3-9 cells to increasing concentrations of the drug (FIG. 8D). Non-linear fit of the individual data points to the Hill equation yielded an IC₅₀ value of 5 μM. The calculated Hill coefficient was ˜1, indicating that channel closure is caused by a simple 1:1 interaction between CBX and Px1 hemi-channels, without cooperativity effect.

Next, the efficacy of CBX to inhibit pannexin and connexin hemi-channels was directly compared. In this series of experiments, it was chosen to use Cx46, because it is efficiently expressed in oocytes and has been extensively studied as the prototype of the hemi-channel forming connexins. After a 30 min incubation in the presence of 10 μM CBX, the I-V relationship of Cx46-expressing oocytes was virtually identical to that measured in control medium, whereas both homomeric Px1 and heteromeric Px1/PX2 hemi-channel currents were drastically decreased by 50-60% (FIG. 9A-C). As in the case of Px1 (FIGS. 8B-C), the effect of CBX on Px1/Px2 channels was fully reversible after washout of the drug and a further 30 min incubation in control medium. Thus, current amplitudes recorded at the +60 mV depolarization step were reduced from 612±50 nA in control medium to 266±24 nA in 10 μM CBX, and then fully recovered to 541±70 nA after the reversibility period (n=6 cells). Comparison of the dose-response data revealed that, in the case of Cx46, the threshold concentration needed for CBX inhibition was higher, whereas the magnitude of the effect was lower even at the largest dose (FIG. 9D-F).

### II-4-Pannexin Hemi-Channels are Relatively Insensitive to Flufenamic Acid

To further explore the pharmacological properties of pannexins, the effect of flufenamic acid (FFA), a member of a large group of chloride channel blockers (64; 65) that has been recently shown to inhibit both connexin hemi- and intercellular channels (66; 67; 68), was determined. Incubation of oocytes (30 min) with increasing FFA concentrations resulted in a modest inhibition of Px1 hemi-channels only at the highest dose (FIG. 10A). By contrast, Cx46 currents were reduced in a dose-dependent manner that resulted in an almost complete blockade with 300 μM FFA (FIG. 10B). It should be noted that higher concentrations of this drug could not be used with confidence, as they often induced unspecific effects (membrane depolarization, large holding currents) that prevented a systematic investigation of their effects.

A more detailed analysis was obtained by comparing the I/V curves of Px1 and Cx46 in the presence of intermediate FFA concentrations. Following a 30 min application of 30 μM FFA, a strong (P<0.005) and fully reversible inhibition of Cx46 hemi-channels was observed, starting with the 0 mV depolarization step, whereas only a weak (10-15%) effect at the more positive potentials was recorded for Px1 currents. Furthermore, dose-response experiments indicated that FFA was much less effective on Px1 hemi-channels, which showed a higher threshold dose than Cx46 and a reduced extent of channel blockade, not exceeding 33% at the highest FFA concentration used (FIG. 10C-D). In keeping with the Px1 results, when FFA was tested on heteromeric Px1/Px2 hemi-channels, a similar weak sensitivity was found only at the largest depolarization steps. Thus, current amplitudes recorded at +60 mV were inhibited by about 15% with 30 μM FFA (from 603±123 nA in MB to 504±101 nA in 30 μM FFA, n=12; P<0.02) and 27% with the highest FFA concentration (from 609±135 nA to 448±104 nA in 300 μM FFA, n=11; P<0.02).

### II-5-Carbenoxolone and Flufenamic Acid Inhibit Pannexin Hemi-Channels via Different Mechanisms

Inspection of the pannexin currents recorded following treatment with CBX and FFA revealed further characteristics that distinguished the mode of action of these drugs.

For example, when the kinetics of Px1 hemi-channel closure in the presence of two concentrations of CBX (3 μM) and FFA (300 μM) that caused approximately the same percentage inhibition (30-35%) of peak current values were compared, obvious differences became apparent. Carbenoxolone induced a decrease in the peak amplitude and a major change in the rectification component that reached a novel steady-state during the duration of the voltage step (FIG. 11A-B), whereas FFA did not affect the kinetic of voltage gating, which remained indistinguishable from that recorded in control medium (FIG. 11E-F). Fitting these traces to a first order exponential decay showed that the time constants (τ) of channel closure were approximately 10 times faster in the presence of CBX (FIG. 11C-D) but did not show any appreciable change with FFA (FIG. 11G-H). Similar results were observed also with other concentrations of CBX (5 and 10 μM) and with Px1/Px2 heteromeric channels (data not shown).

### Notes: Abbreviations

Px, pannexin with the gene number as specified; Cx, connexin with the molecular mass in kDa as specified; βGA, β-glycyrrhetinic acid; BSA, bovine serum albumin; CBX, carbonoxolone; DMEM, Dulbeco's Modified Eagle Medium; EGFP, Enhanced Green Fluorescent Protein; FCS, fetal calf serum; FFA, flufenamic acid; HEK293, Human Embryonic Kidney 293 cells; IC₅₀, inhibitory concentration causing 50% of the effect; IP, immuneprecipitation; Iₘ, membrane current; I-V, current-voltage; MB, Modified Barth's medium; NEM, N-ethyl-maleimide; SDS, sodium dodecyl sulphate; Vₘ, membrane potential.

### REFERENCES

1. Willecke, K., Eiberger, J., Degen, J., Eckardt, D., Romualdi, A., Guldenagel, M., Deutsch, U. & Sohl, G. (2002) Biol Chem 383, 725-37.
2. Bennett, M. V. (2000) Brain Res Brain Res Rev 32, 16-28.
3. Galarreta, M. & Hestrin, S. (2001) Nat Rev Neurosci 2, 425-33.
4. Katsumaru, H., Kosaka, T., Heizmann, C. W. & Hama, K. (1988) Exp Brain Res 72, 363-70.
5. Galarreta, M. & Hestrin, S. (1999) Nature 402, 72-5.
6. Gibson, J. R., Beierlein, M. & Connors, B. W. (1999) Nature 402, 75-9.
7. Beierlein, M., Gibson, J. R. & Connors, B. W. (2000) Nat Neurosci 3, 904-10.
8. Tamas, G., Buhl, E. H., Lorincz, A. & Somogyi, P. (2000) Nat Neurosci 3, 366-71.
9. Venance, L., Rozov, A., Blatow, M., Bumashev, N., Feldmeyer, D. & Monyer, H. (2000) Proc Natl Acad Sci USA 97, 10260-5.
10. Meyer, A. H., Katona, I., Blatow, M., Rozov, A. & Monyer, H. (2002) J Neurosci 22, 7055-64.
11. Blatow, M., Rozov, A., Katona, I., Hormuzdi, S. G., Meyer, A. H., Whittington, M. A., Caputi, A. & Monyer, H. (2003) Neuron 38, 805-17.
12. Ylinen, A., Bragin, A., Nadasdy, Z., Jando, G., Szabo, I., Sik, A. & Buzsaki, G. (1995) J Neurosci 15, 3046.
13. Draguhn, A., Traub, R. D., Schmitz, D. & Jefferys, J. G. (1998) Nature 394, 189-92.
14. Traub, R. D. & Bibbig, A. (2000) J Neurosci 20, 2086-93.
15. Hormuzdi, S. G., Pais, I., LeBeau, F. E., Towers, S. K., Rozov, A., Buhl, E. H., Whittington, M. A. & Monyer, H. (2001) Neuron 31, 487-95.
16. McBain, C. J. & Fisahn, A. (2001) Nat Rev Neurosci 2, 11-23.
17. Traub, R. D., Pais, I., Bibbig, A., LeBeau, F. E., Buhl, E. H., Hormuzdi, S. G., Monyer, H. & Whittington, M. A. (2003) Proc Natl Acad Sci USA 100, 1370-4.
18. Condorelli, D. F., Belluardo, N., Trovato-Salinaro, A. & Mudo, G. (2000) Brain Res Brain Res Rev 32, 72-85.
19. Deans, M. R., Gibson, J. R., Sellitto, C., Connors, B. W. & Paul, D. L. (2001) Neuron 31, 477-85.
20. Guldenagel, M., Ammermuller, J., Feigenspan, A., Teubner, B., Degen, J., Sohi, G., Willecke, K. & Weiler, R. (2001) J Neurosci 21, 6036-44.
21. Deans, M. R., Volgyi, B., Goodenough, D. A., Bloomfield, S. A. & Paul, D. L. (2002) Neuron 36, 703-12.
22. Landisman, C. E., Long, M. A., Beierlein, M., Deans, M. R., Paul, D. L. & Connors, B. W. (2002) J Neurosci 22, 1002-9.
23. Long, M. A., Deans, M. R., Paul, D. L. & Connors, B. W. (2002) J Neurosci 22, 10898-905.
24. Buhl, D. L., Harris, K. D., Hormuzdi, S. G., Monyer, H. & Buzsaki, G. (2003) J Neurosci 23, 1013-8.
25. Panchin, Y., Kelmanson, I., Matz, M., Lukyanov, K., Usman, N. & Lukyanov, S. (2000) Curr Biol 10, R473-4.
26. Wisden, W. & Morris, B. J. (2002) Int Rev Neurobiol 47, 3-59.
27. Emsberger, U., Patzke, H. & Rohrer, H. (1997) Mech Dev 68, 115-26.
28. Groves, J. D. & Tanner, M. J. (1992) J Biol Chem 267, 22163-70.
29. White, T. W., Deans, M. R., O'Brien, J., Al-Ubaidi, M. R., Goodenough, D. A., Ripps, H. & Bruzzone, R. (1999) Eur J Neurosci 11, 1883-90.
30. Barrio, L. C., Suchyna, T., Bargiello, T., Xu, L. X., Roginski, R. S., Bennett, M. V. & Nicholson, B. J. (1991) Proc Natl Acad Sci USA 88, 8410-4.
31. Bruzzone, R., Gomes, D., Denoyelle, E., Duval, N., Perea, J., Veronesi, V., Weil, D., Petit, C., Gabellec, M-M., D'Andrea, P. & White, T. W. (2001) Cell Commun Adhes 8, 425-31.
32. White, T. W., Srinivas, M., Ripps, H., Trovato-Salinaro, A., Condorelli, D. F. & Bruzzone, R. (2002) Am J Physiol Cell Physiol 283, C960-70.
33. Phelan, P. & Starich, T. A. (2001) Bioessays 23, 388-96.
34. Johnston, M. F. & Ramon, F. (1981) Biophys J 39, 115-7.
35. Verselis, V. K., Bennett, M. V. & Bargiello, T. A. (1991) Biophys J 59, 114-26.
36. Kelmanson, I. V., Shagin, D. A., Usman, N., Matz, M. V., Lukyanov, S. A. & Panchin, Y. V. (2002) Eur J Neurosci 16, 2475-6.
37. Paul, D. L., Ebihara, L., Takemoto, L. J., Swenson, K. I. & Goodenough, D. A. (1991) J Cell Biol 115, 1077-89.
38.5 tebbings, L. A., Todman, M. G., Phelan, P., Bacon, J. P. & Davies, J. A. (2000) Mol Biol Cell 11, 2459-70.
39. Brunzzone, R., White, T. W. & Paul, D. L. (1996) Eur J Biochem 238, 1-27.
40. Schmitz, D., Schuchmann, S., Fisahn, A., Draguhn, A., Buhl, E. H., Petrasch-Parwez, E., Dermietzel, R., Heinemann, U. & Traub, R. D. (2001) Neuron 31, 831-40.
41. Traub, R. D., Bibbig, A., Piechofta, A., Draguhn, R. & Schmitz, D. (2001) J Neurophysiol 85, 1246-56.
42. Ebihara, L., Beyer, E. C., Swenson, K. I., Paul, D. L. & Goodenough, D. A. (1989) Science 243, 1194-5.
43. Srinivas, M., Rozental, R., Kojima, T., Dermietzel, R., Mehler, M., Condorelli, D. F., Kessler, J. A. & Spray, D. C. (1999) J Neurosci 19, 9848-55.
44. Al-Ubaidi, M. R., White, T. W., Ripps, H., Poras, I., Avner, P., Gomes, D. & Bruzzone, R. (2000) J Neurosci Res 59, 813-26.
45. Horwell et al., Bioorg. Med. Chem. 4:1573 (1996).
46. Liskamp et al., Recl. Trav. Chim. Pays-Bas 1: 113 (1994).
47. Gante et al., Angew. Chem. Int. Ed. Engi. 33: 1699 (1994).
48. Seebach et al., Helv. Chim. Acta 79: 913 (1996).
49. Mitropoulou et al. J Neurosci Res, 72, 147-157 (2003).
50. Zoidl, G., Bruzzone, R., Weickert, S., Kremer, M., Zoidl, C., Mitropoulou, G., Srinivas, M., Spray, D. C. & Dermietzel, R. J Biol Chem, 279, 2913-2921 (2004).
51. Bruzzone, R., Haefliger, J. A., Gimlich, R. L. & Paul, D. L. Mol Biol Cell, 4, 7-20 (1993).
52. VanSlyke, J. K. & Musil, L. S. Methods Mol Biol, 154, 117-134 (2001).
53. Ebihara, L. & Steiner, E. J Gen Physiol, 102, 59-74 (1993).
54. White, T. W., Deans, M. R., O'Brien, J., Al-Ubaidi, M. R., Goodenough, D. A., Ripps, H. & Bruzzone, R. Eur JNeurosci, 11, 1883-1890 (1999).
55. Li, H., Liu, T. F., Lazrak, A., Peracchia, C., Goldberg, G. S., Lampe, P. D. & Johnson, R. G. J Cell Biol, 134, 1019-1030 (1996).
56. Zampighi, G. A., Loo, D. D., Kreman, M., Eskandari, S. & Wright, E. M. J Gen Physiol, 113, 507-524 (1999).
57. Quist, A. P., Rhee, S. K., Lin, H. & Lal, R. J Cell Biol, 148, 1063-1074 (2000).
58. Ebihara, L., Liu, X. & Pal, J. D. Biophys J, 84, 277-286 (2003).
59. Tran Van Nhieu, G., Clair, C., Bruzzone, R., Mesnil, M., Sansonetti, P. & Combettes, L. Nat Cell Biol, 5, 720-726 (2003).
60. Davidson, J. S. & Baumgarten, I. M. J Pharmacol Exp Ther, 246, 1104-1107 (1988).
61. Rozental, R., Srinivas, M. & Spray, D. C. Methods Mol Biol, 154, 447-476 (2001).
62. Harris, A. L. Q Rev Biophys, 34, 325-472 (2001).
63. Rouach, N., Segal, M., Koulakoff, A., Giaume, C. & Avignone, E. J Physioil, 553, 729-745 (2003).
64. Di Stefano, A., Wittner, M., Schlatter, E., Lang, H. J., Englert, H. & Greger, R. Pflugers Arch, 405 Suppl 1, S95-100 (1985).
65. Schultz, B. D., Singh, A. K., Devor, D. C. & Bridges, R. J. Physiol Rev, 79, S109-144 (1999).
66. Harks, E. G., de Roos, A. D., Peters, P. H., de Haan, L. H., Brouwer, A., Ypey, D. L., van Zoelen, E. J. & Theuvenet, A. P. J Pharmacol Exp Ther, 298, 1033-1041 (2001).
67. Eskandari, S., Zampighi, G. A., Leung, D. W., Wright, E. M. & Loo, D. D. J Membr Biol, 185, 93-102 (2002).
68. Srinivas, M. & Spray, D. C. Mol Pharmacol, 63, 1389-1397 (2003).
69. Arellano J I, Munoz A, Ballesteros-Yanez I, Sola R G, DeFelipe J. (2004) Brain 127, 45-64.

1. Use of at least one pannexin, or at least one biologically active fragment thereof, or at least one biologically active derivative thereof, for the manufacture of a drug for preventing and/or treating, in a mammal, a neurological disorder.

2. The use of claim 1, wherein said pannexin is a neuronal channel-forming pannexin.

3. The use of claim 1 or 2, wherein said neurological disorder involves brain cells.

4. The use of any of claims 1 to 3, wherein said neurological disorder involves hippocampal pyramidal cells.

5. Use of at least one pannexin for in vitro diagnosing, in a mammal, a neurological disorder.

6. The use of claim 5, wherein said pannexin is a neuronal channel-forming pannexin.

7. The use of claim 5 or 6, wherein said neurological disorder involves brain cells.

8. The use of any of claims 5 to 7, wherein said neurological disorder involves hippocampal pyramidal cells.

9. The use according to any of claims 5 to 8, wherein said in vitro diagnostic comprises at least:
a) sequencing a pannexin gene in a mammal suspected to have a neurological disorder; and
b) identifying in said mammal at least one mutation responsible for the lack of production of pannexin or for the production of a pannexin the activity of which is modified compared to a control.

10. The use according to any of claims 1 to 9, wherein said mammal is a human.

11. The use according to any of claims 1 to 10, wherein said neurological disorder is selected from epilepsy, schizophrenia, memory disorders, Alzheimer's disease, pain disorders, visual deficits, visual acuity, odor discrimination, and olfaction deficits.

12. Method for in vitro selecting a compound useful for preventing and/or treating, in a mammal, a neurological disorder, said compound being capable of modifying the channel-forming ability of a pannexin, wherein said method comprises at least:
a) measuring the channel-forming ability of said pannexin in the absence of said compound (P0);
b) measuring the channel-forming ability of said pannexin in the presence of said compound (P1);
c) comparing P0 and P1; and
d) if P1 is significantly different from P0, selecting said compound.

13. Method for in vitro selecting a compound useful for preventing and/or treating, in a mammal, a neurological disorder, said compound being capable of specifically modifying the channel-forming ability of a pannexin, without modifying the channel-forming ability of a connexin, wherein said method comprises at least:
a) measuring the channel-forming ability of each of said pannexin (P0) and said connexin (C0) in the absence of said compound;
b) measuring the channel-forming ability of each of said pannexin (P1) and said connexin (C1) in the presence of said compound;
c) comparing P0 and P1, and C0 and C1; and
d) if P1 is significantly different from P0, and if C1 is not significantly different from C0, selecting said compound.

14. The method according to claim 12 or 13, wherein, in step d), if P1 is significantly greater than P0, said compound is an agonist of said pannexin.

15. The method according to claim 12 or 13, wherein, in step d), if P1 is significantly lower than P0, said compound is an antagonist of said pannexin.

16. The method according to any of claims 12 to 15, further comprising purifying said compound.

17. The method according to any of claims 12 to 16, wherein said mammal is a human.

18. The method according to any of claims 12 to 17, wherein said neurological disorder is selected from epilepsy, schizophrenia, memory disorders, Alzheimer's disease, pain disorders, visual deficits, visual acuity, odor discrimination, and olfaction deficits.

19. Method for in vitro selecting a compound capable of modulating the size of a channel formed by a pannexin, comprising at least the steps of:
a) comparing the movements, between a cell and the medium, of at least one control compound, the molecular size of which is known, in the presence and in the absence of a candidate compound; and
b) if a difference in said movements is observed, selecting said candidate compound.

20. An animal model for in vivo selecting a compound useful for preventing and/or treating, in a mammal, a neurological disorder, wherein said compound is capable of modifying the channel-forming ability of a pannexin in said animal model.

21. The animal model according to claim 20, wherein at least one mutation is introduced in a pannexin gene of an animal, said mutation being responsible for the lack of production of pannexin, or for the production of a totally or partially inactive pannexin, said totally or partially inactive pannexin exhibiting a reduced or suppressed channel-forming activity, in said animal model.

22. The animal model according to claim 20, wherein a reporter gene is introduced under the control of the endogenous promoter of a pannexin gene, into the genome of said animal model.

23. The animal model according to claim 20 to 22, wherein said mammal is a human.

24. The animal model according to any of claims 20 to 23, wherein said neurological disorder is selected from epilepsy, schizophrenia, memory disorders, Alzheimer's disease, pain disorders, visual deficits, visual acuity, or discrimination, and olfaction deficits.

25. Use of an animal model according to any of claims 20 to 24 for in vitro selecting a compound capable of modifying the channel-forming ability of a pannexin.


SEQUENCE LISTING (18 sequences)

SEQ ID NO: 1 - DNA, 2782 bp, Homo sapiens
gggcggcgcggaggggcagggccagagggaagcgctttgttccgcgcgtggttcccgcgcctgggggtgcgcgggagaggcgcgaatccgagtgccgcgcgcggcccggggacttgcacgggcgtgcggggtggaaccgcaggaagcggagctctcgggttcccgccccgccccgccccgccggcggcggaggcagcgagcgcgagagcccagcggagtcgctgggagcctgaggcaccgagacacaaaggcaggcgggatgcgggagcaggcaaagggaaagcgaaagccgcgcgcccggccggtgactgggtgaaggcgccgcgcagctttcccgacgccggctgtacccggacctcctggtcgagcctggcgcgccgcagccatggccatcgctcaactggccacggagtacgtgttctcggatttcttgctgaaggagcccacggagcccaagttcaaggggctgcgactggagctggctgtggacaagatggtcacgtgcattgcggtggggctgcccctgctgctcatctcgctggccttcgcgcaggagatctcgattggtacacagataagctgtttctctccaagttctttctcctggcgtcaggctgcctttgtggattcatattgctgggcggctgttcagcagaagaactcactgcagagcgagtctggaaacctcccactgtggctgcataagtttttcccctacatcctgctgctctttgcgatcctcctgtacctgcccccgctgttctggcgtttcgcagctgctcctcatatttgctcagacttgaagtttatcatggaagaacttgacaaagtttacaaccgtgcaattaaggctgcaaagagtgcgcgtgaccttgacatgagagatggagcctgctcagttccaggtgttaccgagaacttagggcaaagtttgtgggaggtatctgaaagccacttcaagtacccaattgtggagcagtacttgaagacaaagaaaaattctaataatttaatcatcaagtacattagctgccgcctgctgacactcatcattatactgttagcgtgtatctacctgggctattacttcagcctctcctcactctcagacgagtttgtgtgcagcatcaaatcagggatcctgagaaacgacagcaccgtgcccgatcagtttcagtgcaaactcattgccgtgggcatcttccagttgctcagtgtcattaaccttgtggtttatgtcctgctggctcccgtggttgtctacacgctgtttgttccattccgacagaagacagatgttctcaaagtgtacgaaatcctccccacttttgatgttctgcatttcaaatctgaagggtacaacgatttgagcctctacaatctcttcttggaggaaaatataagtgaggtcaagtcatacaagtgtcttaaggtactggagaatattaagagcagtggtcaggggatcgacccaatgctactcctgacaaaccttggcatgatcaagatggatgttgttgatggcaaaactcccatgtctgcagagatgagagaggagcaggggaaccagacggcagagctccaaggtatgaacatagacagtgaaactaaagcaaataatggagagaagaatgcccgacagagacttctggattcttcttgctgatgatttttttccttgagctgtaaatctgtgacttctgcgacatgggatttaatttggctaaagcacccctgttggtttcacagctggtttgcaataaatggttcttggtggagatactgagcatgtcttattgagtccctaatggaaatggtgatcaacaaaaggttatggaagaatggtttatgaacttcccataggaagcacctgagagatagtaaactgcagcaagtaactatgtgtaagtcctcatcaaatgaaaagcagaaagacaagaacaattagtcaagagcagtagccctgtcagagcctcggagcaatacctttctgtacccgtggtgagacaagacccagagctactggaaaacaagcactttggaagatttgttttgttttcatggaataataatatgtcagggtataatttaacgtgagtttcttatgtgcccttaaagactgttagacaagaaaagcattcactggctaataatccataggtcgacctatgtcctaagttaggtgtaaggtccgatgccttggcccacactcgagctctctttacattgttagttgtcaaccttggctgatggaaatcccgtaaccactatttgttgcactgtgccttgaagggcagcaggcccaagtgctgctctgactgaaaactgagttaacaagatgaaatctaaaggatattcacagtgacttcaattcaggaagaatgcttccaaaagagcccagtggggaaatctgacatcacagaagacattaattcagtcactttcaaagagtttgtctacaggcggtttctctgttatcaaaggcatttgaaataggattttacttaaacaataatggaacacaggagtatttaaagtgaagaacactttgcctgaatgtgatcagggcacataagtgacattggcatgcttcatatggcgtgcttggagccagaaaaacttagcggtttattttgtttatatttaagcacagctttaaaaaattcattatcgtttattcagtgtccgaattgaggccatttgggaagaaaattctagcactggtggagaattatagaataaagattataaatggttggataagacaaaaaaaaaaaaaaaa

SEQ ID NO: 2 - protein, 426 aa, Homo sapiens [sequence omitted]

SEQ ID NO: 3 - DNA, 2950 bp, Homo sapiens
atggcgaccgcgctgctggcgggagagaagctgcgggagctgatcctgccgggcgcgcaggacgacaaggcgggcgcgctggccgcgctgcttctgcagctgaagctggagctgccgttcgaccgggtggtcaccatcggcaccgtgctggtgcccatcctgctggtcaccctggtcttcaccaagaacttcgcagaggaacccatttactgttacaccccgcacaacttcacgcgcgaccaggcgctgtacgcccgcggctactgctggacggagctgcgggacgcgctgcccggcgtggacgccagcctgtggccgtcgctgtttgagcacaagttcctgccctacgcgctgctggccttcgccgccatcatgtacgtgcccgcgctgggctgggagttcctggcctccacgcgcctcacctccgagctcaacttcctgctgcaggagatcgacaactgttaccaccgggcggccgagggccgcgcgcccaagatcgagaagcagatccagtccaagggcccgggcatcacggagcgcgagaagcgcgagatcatcgagaacgcggagaaggagaagagcccggagcagaacctgttcgagaagtacctggagcgccgcggccgcagcaacttcctggccaagctgtacctggcgcggcacgtgctgatcctgctgctgagcgccgtgcccatctcctacctgtgcacctactacgccacgcagaagcagaacgagttcacctgcgcgctgggcgcgtccccggacggggcggcaggtgcggggcccgcggtgcgcgtgagctgcaagctcccgtccgtgcaactgcagcgcatcatcgcgggcgtggacatcgtgctgctgtgcgtcatgaacctcatcatcctcgtcaacctcatccacctcttcatcttccgcaagagcaacttcatcttcgacaagctgcacaaggtgggcatcaagacgcgccggcagtggcgccgctcgcagttctgcgacatcaacatcctggccatgttctgcaacgagaaccgcgaccacatcaagtcgctcaaccggctggacttcatcaccaacgagagcgacctcatgtacgacaacgtggtccggcagctgctggcggcgctggcgcagtccaaccacgacgccacccccacggtgcgcgactcgggggtgcagaccgtggaccccagcgccaaccccgccgagcccgacggcgccgccgagccgcccgtggtcaagcggccgcgcaagaagatgaagtggatccccaccagcaacccgcttccgcagcccttcaaggagccgctggccatcatgcgcgtggagaacagcaaggcggagaagccgaagcccgcgcgcaggaagacggccacggacacgctgatcgcgccgctgctggaccgctccgcccaccactacaagggcggagggggcgacccgggccccggccccgcccctgcccccgccccgccgcccgcccctgacaagaagcacgcgcgccacttctccctggacgtgcacccctacatcctcggcaccaagaaggccaaggccgaggcggtgcccgccgccctgcccgcctcccggagccaggaggggggcttcctgtcccaggcggaggactgtgggctaggcctggccccggcgcccatcaaagatgctccgctccccgagaaggaaatcccgtaccccacagagccagcccgggcagggcttccctcggggggcccgttccacgtccgctcacctcccgccgcccctgctgtggcccctctgacaccagccagcctgggcaaggcggagcccctcaccatcctgagccgaaacgccacacacccgctgctgcacatcaacacgctatcctcatcgccaccttcgacgagccgagaacggtcgtgagtactgtggagttttgagggatggcaccgtccaggccgccgagagcccctctgcctgtgtcgtgtggcctggccagcctcccggtggacaccagccctgcgtggacgtggcctgtgcttcgcccgcactgcgcgcatccccaacctctgtccgcatgcctggggccttcgcccccacgtgctcgacaggggaacccgcccggacggcatcgccaggcactggctggggtggggaaaggtggcccagtggagccggtggccaggaaggctgaagcccgcttcccatgctcctgcatcaggtgcccagccgtgggtgggggccctgaggtgaagagtttatttttttagtccgtttcgtcctggccccgggctgtggcgagacagcccaactcccccagcccagctcccccagcccagagccagggaagaggaaggtggggccagtcccaccagtggggtggccacgcccatggggtcacatgctcaggggtcaccccctgcagggacctgatgccctcgggtgggagggaccgaggtccaccctcgggtcaaaggtcaacgtgcactttctccttgtcgcctgacagacattttattttactaagactgctgtaccgaacaagcatatttatcatcaggagacaggatgggtttaaagcaggatggtgtgtgtgtgaacgggcatgagcagaggtgagcgtgagcgagcgggtgtgtatgtacgagtgtgcacgtgtgtgcgtgtgcacagagggtgtggtgccagcttgagtgggagtgtgtgagtgtgagcaggcgggcgagtgcgtgagtgcacgccagcgcgtggcccatgtatgaggagtgaaggggcccaacgcaataaccacgtcccccacccgggccccccgccgcggctgaggccacatggcttcctgtgggagccccggccggcacccggctggtcccaccccaaatacctcagccatggagaccatgtcatgcagaattaacaaggtagcaccgagcatatcaataaatattattctgataaaaaaaaaaaaaaaaaa

SEQ ID NO: 4 - protein, 633 aa, Homo sapiens
MATALLAGEKLRELILPGAQDDKAGALAALLLQLKLELPFDRVVTIGTVLVPILLVTLVFTKNFAEEPIYCYTPHNFTRDQALYARGYCWTELRDALPGVDASLWPSLFEHKFLPYALLAFAAIMYVPALGWEFLASTRLTSELNFLLQEIDNCYHRAAEGRAPKIEKQIQSKGPGITEREKREIIENAEKEKSPEQNLFEKYLERRGRSNFLAKLYLARHVLILLLSAVPISYLCTYYATQKQNEFTCALGASPDGAAGAGPAVRVSCKLPSVQLQRIIAGVDIVLLCVMNLIILVNLIHLFIFRKSNFIFDKLHKVGIKTRRQWRRSQFCDINILAMFCNENRDHIKSLNRLDFITNESDLMYDNVVRQLLAALAQSNHDATPTVRDSGVQTVDPSANPAEPDGAAEPPVVKRPRKKMKWIPTSNPLPQPFKEPLAIMRVENSKAEKPKPARRKTATDTLIAPLLDRSAHHYKGGGGDPGPGPAPAPAPPPAPDKKHARHFSLDVHPYILGTKKAKAEAVPAALPASRSQEGGFLSQAEDCGLGLAPAPIKDAPLPEKEIPYPTEPARAGLPSGGPFHVRSPPAAPAVAPLTPASLGKAEPLTILSRNATHPLLHINTLSSSPPSTSRERS

SEQ ID NO: 5 - DNA, 1609 bp, Homo sapiens
atgtcacttgcacacacagctgcagagtacatgctctcagatgccctgctgcctgaccgcaggggaccccgcctcaaaggactgcgtctggaactgcccctggaccggatagtcaagttcgtagctgtgggctcccccttgttgctgatgtccctggcattcgcccaggagttctcctctgggtctccgatcagctgcttctctcccagtaacttcagcatccggcaggcagcctacgtggacagctcctgctgggactcactgcttcaccataagcaggacgggcctggccaggacaaaatgaaatctctctggccccacaaggccctcccctactccctgctggccctggccttgctcatgtacctgccggtgctgctgtggcagtatgcagctgtgccagccctcagctccgatctgctgttcatcatcagcgaactggacaaatcttataatcgctccatccgcctcgtgcagcacatgctgaagatccggcagaagagttccgacccctatgtgttctggaatgagctggagaaggctcggaaagaacgatactttgaattccctttgctagagcggtacctggcatgtaagcagcgttcacattcgctagtggctacctacctcctgaggaactccctcttgctcatcttcacctccgccacttacctataccttggtcatttccatctggatgtcttcttccaggaagaattcagctgctccatcaagacagggctgctaagtgatgagacccatgtccccaatctgatcacatgcaggctgacatcactgtccattttccagattgttagcctctccagtgtagcaatatacaccatattggttccagtgataatatacaacctcacacggctatgtcggtgggacaaacgacttttatctgtctatgagatgctcccagcttttgatctcctcagcagaaagatgctaggatgtcccatcaatgacctcaatgtgatccttcttttcctccgagctaacatctctgagctcatctcttttagctggctgagtgtcttatgtgtgttgaaggatacaaccacccagaagcacaatattgacacagtagttgattttatgactttattggctggcttagaaccctcaaaacccaaacacctcaccaactcggcatgtgatgaacacccatagttaagaaaccatggagcaagaaagcttgtggaaagtctctctccttcctcataagacatgcacactaatacacatacacaccaaaaaattacacattttaaaactgctaagcttggatttaactgaatcatatatcttttatcatgttatcctaaaagtgagaagacataaccaagacatggaaataaatgtgaaagctggagccgaagagtcaaagagctaaaaaattaagtctagaacattctatgaggatagtataaataaaaagaaatacagtctagacatgctgcaaggaaagaagattctaaagtccgtttatggaggcaattccatatcctttcttgaacgcacattcagcttaccccagagagcaagtgaggcaatctggcaaaagattaataaagatgtaaacccctggaaaaaaaaaaaa

SEQ ID NO: 6 - protein, 392 aa, Homo sapiens
MSLAHTAAEYMLSDALLPDRRGPRLKGLRLELPLDRIVKFVAVGSPLLLMSLAFAQEFSSGSPISCFSPSNFSIRQAAYVDSSCWDSLLHHKQDGPGQDKMKSLWPHKALPYSLLALALLMYLPVLLWQYAAVPALSSDLLFIISELDKSYNRSIRLVQHMLKIRQKSSDPYVFWNELEKARKERYFEFPLLERYLACKQRSHSLVATYLLRNSLLLIFTSATYLYLGHFHLDVFFQEEFSCSIKTGLLSDETHVPNLITCRLTSLSIFQIVSLSSVAIYTILVPVIIYNLTRLCRWDKRLLSVYEMLPAFDLLSRKMLGCPINDLNVILLFLRANISELISFSWLSVLCVLKDTTTQKHNIDTVVDFMTLLAGLEPSKPKHLTNSACDEHP

SEQ ID NO: 7 - DNA, 2903 bp, Rattus norvegicus
cccggtttcccccgcgtaggttccggaagagctcggttcccgtagccgagcgcgcagtgcaggcgcgagcagctgtagcggagagctagctttgttcccggcacgttgttgtccctgcgcgctcaaggcggccggagtcctggtgtcgcgcgcgctggggacccgcgggagctgagcacggggtgtgatcgctgccgccaagacttcggggctcgtgcgccctgtgttgcttggagcgatcccaggcggctgcagctctcgtgcggtccctgcgaggtaggcgcagcgactgccaaggcggtgcaatgctgggccggcccggaagccgcgctccgggtcgttgacggcgcggactcggcgcctctccccggcttcccgcagcacccgcgcctgccttcgactgcccgtcccgcggcacccggcgcgccgtgaccatggccatcgcccacctggccacggagtacgtgttctcggacttcttgctgaaggagcccaccgagcccaagttcaaggggctgcgactggagctggcagtggacaagatggtcacgtgcattgccgtgggtctgcctctgctgctcatctcgctggccttcgctcaggagatctccatcggtacccagataagctgcttctccccaagttccttctcctggcgccaggccgccttcgtggactcttactgctgggccgctgtacagcagaaaaactccctgcagagtgagtctggaaacctcccgctgtggctgcacaagttcttcccctacatcctgctactgtttgccatactcctgtacctgcccgctctgttctggcgctttgcggcggctccacacctctgctccgacctgaagtttatcatggaggagcttgacaaagtctataaccgcgccatcaaggctgccaagagtgctcgtgatttggacctgagagatggacctggacctccaggagtgactgagaatgtggggcagagtctgtgggagatatccgaaagccacttcaagtacccgatcgtggagcagtacttgaagacgaagaagaactccagtcacctaatcatgaaatacattagctgccggctggtgacctttgcggtggtactgctggcttgcatctacttgagctactacttcagcctctcctcgctctccgacgagtttctgtgtagcatcaagtcgggcgtcctgaggaacgacagcaccatccccgatagcttccagtgcaagctcatcgcggtcggcatcttccagctgctcagcctcattaacctcctagtgtacgccctgctggtccccgtggtcatctacacgctcttcgtcccgttccggcagaagacggacgtcctcaaggtatacgagatcctgcccaccttcgatgttctacatttcaagtctgaaggctacaacgacttgagcctctacaacctttttctggaagagaacataagtgagctcaaatcgtacaagtgtcttaaggtgctggagaacattaagagcaatgggcagggcatcgaccccatgttacttctgacaaacctaggcatgattaagatggacgtcattgatggaaaagtccccatgtccctacagaccaagggagaggaccagggcagccagagaatggacttcaaagatttggacctgagcagcgagacggcagcgaacaatggggagaagaactctcgccagcgacttctgaattcatcctgctaatggtttcattcttgaatttcaagcctgtgactcctgtggctgacccacccctgttggaaccgtagctagtttgcagtatatgtttttggtatcgatgtggtgtgtgtcctaagacataatggcccacagcgttatggaagaatagctaggaacgtccttcaagactaaatatggaaacgagtaagtgctttgtctaaagccctcgtcggagggctattaagagagagcacagagccttctagagccccagagaagcaccgttctggactggcagtgctgggagacctggagcttctagagaacaagcactttgttttgtgagataataataaacctgtcaggatgtaatttaataaccccttctgtgtgccctttctgactgttggacaagaagagcattcactggcctaccagggaaatcgacctgttcgagtgcagctgtgccctaaattgggtgtgacctgccgtttgacctcagcacggacagcagcctgaggcagtgctgactaaaccccagctatggagccagagtcatgccatggtgactctcagaaggaaggcttgagaaaccttcccagtgggaaacctgagtttagatacctgaggcaggagaacgggattttactgacgcccatgtgaggcacactttgtccaatgagggcagaacacggaactgacctagcagtggccttggccatttgtttagatcagattgattaaatttaagcccaacttttaaaattgcacattgatttatataatcgaagtcatttgagaagagcattccagcactgataagggttttggagtaagtcgtacagtaggaggttagaacagactctggcattgctttgttttgtttgcattgctttgttttgttttgtgacacggccccctgcctcactcagactcagactgccctcagggtcttccttccctcactcttccaagctgacccagaattcgttaaaacctgattacattctgaccacatgagtgtgttacatgttcgcagtttatatagaaatctttagtcttcacaccttcccagttttaccaggtttaaagggaagtcctcaggaacctcccagtttactttgggtcaatcataggaacaattgtttacctaatctaatggcttttggtgaaatcggaagttacgttaagtggttgtctgcctttgggggc

SEQ ID NO: 8 - protein, 426 aa, Rattus norvegicus
MAIAHLATEYVFSDFLLKEPTEPKFKGLRLELAVDKMVTCIAVGLPLLLISLAFAQEISIGTQISCFSPSSFSWRQAAFVDSYCWAAVQQKNSLQSESGNLPLWLHKFFPYILLLFAILLYLPALFWRFAAAPHLCSDLKFIMEELDKVYNRAIKAAKSARDLDLRDGPGPPGVTENVGQSLWEISESHFKYPIVEQYLKTKKNSSHLIMKYISCRLVTFAVVLLACIYLSYYFSLSSLSDEFLCSIKSGVLRNDSTIPDSFQCKLIAVGIFQLLSLINLLVYALLVPVVIYTLFVPFRQKTDVLKVYEILPTFDVLHFKSEGYNDLSLYNLFLEENISELKSYKCLKVLENIKSNGQGIDPMLLLTNLGMIKMDVIDGKVPMSLQTKGEDQGSQRMDFKDLDLSSETAANNGEKNSRQRLLNSSC

SEQ ID NO: 9 - DNA, 2895 bp, Rattus norvegicus
ggtaaacagcccccctcccaggcgggagcgggccgcgcggcccaggcaggatagccgccgccgcccccgggagggacgcagggcgcgggccagctacagcgcacggcgggggcggcggcggcccggcgccctggcagctcgacccctctcgcggggcggcagacccccgagccccgccgcatccccgctgacatccctcttccagcgccctgcgccccgcgcccccgtgacctcggtctccgtcgccccccgccgccctctctggcgcgcggccccgcccgcgccccccgcccgtaccccgccgggccgcgcccccgccccccatgcaccacctcctggagcagtcggcggacatggcgaccgcgctgctggcgggcgagaagctgagggagctgatcctgcctggctcgcaggacgacaaggcgggtgcgctggccgcgctgctgctgcagctcaagctggaactgccgttcgaccgcgtggtcaccatcggcaccgtgctggtacctatcctgctggtcaccctggtcttcaccaagaacttcgcagaggaaccaatttactgttatactccgcacaacttcacccgtgaccaggcgctgtacgcccgtggctactgctggacagagctgcgggatgccctgcccggcgtggatgccagcctgtggccatcgctgtttgagcacaagttcctgccctacgcactgctggcctttgccgccatcatgtatgtgcccgcactgggctgggagttcctggcctccacgcgcctcacctccgagctcaacttcctgcttcaggagatcgacaactgctaccaccgagccgccgaaggtcgcgcccccaagattgagaagcagatccagtccaaggggccaggcatcacggagcgcgagaagcgggaaatcattgagaacgctgagaaggagaagagccctgagcagaatttgtttgagaagtacctggagcgccggggccgcagcaacttcttggccaagctgtacttggcgcggcacgtactgatcctgctactcagtgtggtgcccatctcttacctatgcacatactacgccacccagaagcagaacgagttcacctgcgccctgggcgcctcgcctgacgggccggtgggtagcgccgggcccacggtgcgcgtcagctgtaagctgccctccgtgcagctgcagcggatcattgcgggtgtggacatcgtattgctctgcttcatgaacctcatcatcctggtcaacctcatccacctcttcatcttccgcaagagcaacttcatttttgacaagctgcacaaggtgggtatcaagacgcgccggcagtggcgccgctcgcagttctgcgacatcaacatcctggccatgttctgtaacgagaaccgtgaccacatcaagtcgcttaaccggctggacttcatcaccaacgagagcgacctcatgtacgacaacgtggtgcggcagcttctggctgccttggctcagtctaaccacgacaccacgcccacggttcgggactcaggcatccagaccgtggaccccagcatcaaccccgcagagcctgagggctctgctgagccacccgtggtcaagcggccccgtaagaaaatgaagtggatccccaccagcaacccgctgccacagcccttcaaggagcagctggccattatgcgcgtagaaaacagcaagactgagaagcccaaacctgtgcgcaggaagacagccacggatacacttattgccccgctgctggacgctggtgcccgggctgcccaccactacaaggggagtggaggtgatacagggccctcctctgccccgcctgctgcctctgagaaaaagcatacccggcacttctccttggatgtgcatccctatatcctaggcagcaagaaggccaagactgaagcagtaccccctgccctaccagcctcccggagccaggaaggtggcttcctgtcccagacagaggaatgtgggctgggcttggctgcagcacccaccaaagatgctccgctccctgagaaggaaatcccataccccacagagtctgccctgccgtctgggggcccattccatgtgtgctcaccccccacggcctctgctgctgcttccctgtcaccgagcagcctgggcaaggctgaccccctcaccatcctgagccgaaacgccactcaccccctgctccacatcagcacgttgtacgaggcccgggaagaggaggaaggaggtccctgtgcaccctcagacatgggcgacctcctcagcatacccccaccccagcagatcctcatcgccaccttcgaggagccgagaacagttgtgagtactgtggagttttgaggggcagggcctcccaggccaccaaaacctctctgcgagagccagggatggcccagctgagcctaggcatgagtagatccgaactctgctcacccagtaccgcccacaacccccagcctcctgtctgcatgccacggggcccagccctccttccacctggctcaccatggcatccacaagtgcaggccggggctcgagcacaaggtgggagagggtgcctaccccagcatcaggcacccccctcagcatgctgggccacaagatgaagagtttatttttttagtgggttttgttgtgggctgacacaactcagcacacccagttgggcccagcctagggtaagtgaggacggtgctggggaaggtggtactggcctcaggacagaccccaggggtcactcacttaagggcctggcccctacagagggacctgataccttcaggtggaggggggactgagggctgccccgggtccaaatcaacatgcactttctccacgtagcctgacacacactttatttttactatgactactgtaactgacgagc

SEQ ID NO: 10 - protein, 664 aa, Rattus norvegicus
MATALLAGEKLRELILPGSQDDKAGALAALLLQLKLELPFDRVVTIGTVLVPILLVTLVFTKNFAEEPIYCYTPHNFTRDQALYARGYCWTELRDALPGVDASLWPSLFEHKFLPYALLAFAAIMYVPALGWEFLASTRLTSELNFLLQEIDNCYHRAAEGRAPKIEKQIQSKGPGITEREKREIIENAEKEKSPEQNLFEKYLERRGRSNFLAKLYLARHVLILLLSVVPISYLCTYYATQKQNEFTCALGASPDGPVGSAGPTVRVSCKLPSVQLQRIIAGVDIVLLCFMNLIILVNLIHLFIFRKSNFIFDKLHKVGIKTRRQWRRSQFCDINILAMFCNENRDHIKSLNRLDFITNESDLMYDNVVRQLLAALAQSNHDTTPTVRDSGIQTVDPSINPAEPEGSAEPPVVKRPRKKMKWIPTSNPLPQPFKEQLAIMRVENSKTEKPKPVRRKTATDTLIAPLLDAGARAAHHYKGSGGDTGPSSAPPAASEKKHTRHFSLDVHPYILGSKKAKTEAVPPALPASRSQEGGFLSQTEECGLGLAAAPTKDAPLPEKEIPYPTESALPSGGPFHVCSPPTASAAASLSPSSLGKADPLTILSRNATHPLLHISTLYEAREEEEGGPCAPSDMGDLLSIPPPQQILIATFEEPRTVVSTVEF

SEQ ID NO: 11 - DNA, 2422 bp, Rattus norvegicus
aagccacagtgcttcgtctgttgggagacaatctaggcaagcaccctggcaccagacacaagtaaagcctgaccctcctccagtgaagacccatcaaggctatgccctcctctaattgccagggcgtctggcttagagcctgggcaattatgtagagaaccactgcctggtactagcacagatctgtgaggctggtgctgcgacatagctgcctgaagctgtcacatcaccccactgtacctagattgctaagagtagcagtgtcctctgtattcccccaaagcccccatactcagcagcatcatgtcactcgcacacacagcggcagagtacatgctctccgatgccctgctgcctgaccgcaggggctctcggctcaaagggctgcgtctggaactgccgctggataagatggtcaagttcgttactgtgggctttcccttgctgctgatgtctctggctttcgcccaggagttctcatcagggtctccgatcagctgcttctctcccagcaacttcagtgtcagacaggctgtgtttgtggacagctcctgctgggactcactggctcactataaacaggatgaggctggacagtacacggtgaaatctctctggcctcacaaggctctcccctactctctactggccctggctgtggccatgtatctgccagtgctgctctggcaatatgcagctgtgccagccctcagctcagacctgctattcatcatcagtgagctggacaagtcttacaaccgctccatccgcctggtacagcacatgctgaagatccggcagaagagctctgaccctcatgtcttctgggatgagctagagaaggcccggaaggaacggtactttgaatttcccttgctagagcggtacctggcatgcaagcagcgctcccattggctagtggccacctacctcctgaggaacgcccttttactcctcttcacctcagccacttacctgtaccttggccacttccatttagacgtcttctttcaggaagaattcagctgctccatcaagacaggcctactccatgaggagacccacgtgccagagctgatcacatgcaggctgacctccctgtctgtattccagattgtgagtgtctccagtgtagcaatatacaccgtactggtcccagtgataatatacaacctcacgcggctgtgccggtgggacaaacgactcctctccatctacgagatgctcccagcttttgatctcctcagtagaaagatgctagggtgccccatcaatgacctcaatgtgatccttcttttcctccgagctaatatctctgagctcatctcttttagctggctgagtgtcttatgtgtgttgaaggatacaaccacccagaaacacaacattgacacagtggtcgattttatgaccttattggctggcttagaaccctcaaaacctaaacatctcacccaacatacgtatgatgaacacccatagtttaaaaaaaaaaagaggcataagagataagttttatagaaagtaagtctccttcttcacaagctatgtagagtaaaagaaaaataataatgatgtctgcattaaattgttaagattagacttaactgaaatatcctgtatttataagaagataaaaatcaatgcatggaagttcaaggtgaagtctgaaagtgaagagtcaaagagctgaaaaatcagtctagagaattctgaggacagtaaacgctaaacgatcaagttgaagcaaagtgcaaacattgaacacagaacaattctgaaggccatttatggaaggaaattctcacaagacccacccactctagaagtcatgcttgcctctgcacaatgggcaggcagagcaacttagtaagagattaataaggatatgaccccctgaaacactttgatttggcctgcccatcctgctttgtggacattttcagccttgccaagcaccccatggcctgctaatgagacaagaaatagtgatggctttaacttgtaatctgctcttggaacataggacagaggcaaaggccacgagttgactctgtaagtcagttacgttttttatgatattaactgaggattcttttccatgagggctcatattatagcctatgaagcccttctaaagtcttaatgagtctaggtatgttcttaatagtccaagggatactacacgttcactgacagccaactagtgaaggccagtgggttagacctgatttcaatgcctgggacccaaatggtgaaaagggagaactgattctccaaggctagcccctgatcgccatatacaaaacaaagtaaacaaataaaaaagtattaagcaagatgaacgtgaaagtcatattgatccacttctgtaattcaccgttcatcttgcttaatacttttttatttgtttactttgttt

SEQ ID NO: 12 - protein, 392 aa, Rattus norvegicus
MSLAHTAAEYMLSDALLPDRRGSRLKGLRLELPLDKMVKFVTVGFPLLLMSLAFAQEFSSGSPISCFSPSNFSVRQAVFVDSSCWDSLAHYKQDEAGQYTVKSLWPHKALPYSLLALAVAMYLPVLLWQYAAVPALSSDLLFIISELDKSYNRSIRLVQHMLKIRQKSSDPHVFWDELEKARKERYFEFPLLERYLACKQRSHWLVATYLLRNALLLLFTSATYLYLGHFHLDVFFQEEFSCSIKTGLLHEETHVPELITCRLTSLSVFQIVSVSSVAIYTVLVPVIIYNLTRLCRWDKRLLSIYEMLPAFDLLSRKMLGCPINDLNVILLFLRANISELISFSWLSVLCVLKDTTTQKHNIDTVVDFMTLLAGLEPSKPKHLTQHTYDEHP

SEQ ID NO: 13 - DNA, 1673 bp, Mus musculus
ctcggctgcccgtcccgcggcacccggtgcgccttgaccatggccatcgcccacttggccacggagtatgtgttctcggacttcttgctgaaggagcccaccgagcccaagttcaaggggctgcgactggagctggcggtggacaagatggtcacatgtattgccgtgggtctacctctgctgctcatctcgctggccttcgctcaggagatctccatcggtacccagataagctgcttctccccgagttctttctcctggcgacaggctgcctttgtggattcatactgctgggctgctgtacagcaaaagagctccctgcagagcgagtctggaaacctcccactgtggctgcacaagttcttcccctacatcctactgctgtttgccatactcctgtacctgcccgcactcttctggcgcttctctgcagctccacacctctgctcagacctgaagtttatcatggaggaacttgacaaagtctacaaccgcgccatcaaggctgccaagagtgctcgagatttggacctaagagacggacctggacccccaggagtgactgagaatgtggggcagagtctgtgggagatatctgaaagccacttcaagtacccaatcgtggagcagtacttgaagacaaaaaagaactctagtcatttaatcatgaaatacattagctgccggctggtgacatttgtggttatactgttggcatgtatctacttgagctattacttcagcctctcttcactctcggacgagtttctgtgcagcatcaaatcaggcgtcctgaaaaatgacagcaccatccccgatcgcttccagtgcaagctcatcgccgtgggcatcttccagctgctcagcctcattaacctcattgtgtatgctctgctgattcccgtggtcgtctacacgttcttcaccccattccggcagaaaacggacattctcaaagtgtatgaaatcctgcccaccttcgatgttctacatttcaagtctgaaggctacaatgacttgagcctctacaacctttttctggaagagaacataagtgagctcaaatcgtacaagtgtctgaaggtgctggagaacattaaaagcaatgggcagggcattgaccccatgctactcctgacaaacctgggcatgattaagatggacatcattgatggaaaaattcccacgtccctacagaccaagggagaggaccagggcagccagagagtggagttcaaagatttggacctgagcagcgaggctcgagcaaacaatggggagaagaactctcgccagaggctgtgtgaatccgtcctgctaatggtttccttcttgaatttcaagcctgtgacttctgtggctgacgcacccctgttggatctgtagttagtttgcagtagatgtttttggtattgatgtagtgtgtgtcctaccaatctctaatagacataatggccaacagcgtcatagaagaatagattagaaacgtcccacaagagtaagtgtgctttgtgtgaagtcctcattgcagggctgttaagagcacagagcctcagccacagaggagcagcattctggactggcaggtgctagaggacctggggcttctagagagcaagcacatggaagatgctttgttttgtgaggtaataaacatgtgaggatgaaactta

SEQ ID NO: 14 - protein, 426 aa, Mus musculus
MAIAHLATEYVFSDFLLKEPTEPKFKGLRLELAVDKMVTCIAVGLPLLLISLAFAQEISIGTQISCFSPSSFSWRQAAFVDSYCWAAVQQKSSLQSESGNLPLWLHKFFPYILLLFAILLYLPALFWRFSAAPHLCSDLKFIMEELDKVYNRAIKAAKSARDLDLRDGPGPPGVTENVGQSLWEISESHFKYPIVEQYLKTKKNSSHLIMKYISCRLVTFVVILLACIYLSYYFSLSSLSDEFLCSIKSGVLKNDSTIPDRFQCKLIAVGIFQLLSLINLIVYALLIPVVVYTFFIPFRQKTDILKVYEILPTFDVLHFKSEGYNDLSLYNLFLEENISELKSYKCLKVLENIKSNGQGIDPMLLLTNLGMIKMDIIDGKIPTSLQTKGEDQGSQRVEFKDLDLSSEARANNGEKNSRQRLLNPSC

SEQ ID NO: 15 - DNA, 1922 bp, Mus musculus
atggcgaccgcgctgctggcgggcgagaagctgagggagctgatcctgcctggctcgcaggacgacaaggcgggcgcgctggccgcgctgctgctgcagctcaagctggaactgccgttcgaccgcgtggtcaccatcggcaccgtgctggtacccatcctgctggtcaccctggtcttcaccaagaacttcgcagaggaaccaatttactgttatactccgcacaacttcacccgtgatcaggcgctgtacgcccgcggctactgctggacagagctgcgggacgcgctgcccggcgtggatgccagcctctggccatcgttgtttgagcacaagttcctgccctacgcgctgctggcctttgccgccatcatgtatgtgcccgcgctgggctgggagttcctcgcctccacgcgcctcacctcggagctcaacttccttcttcaggagatcgacaactgctaccaccgagcggccgaaggtcgcgcacccaagattgagaagcagatccagtccaaggggcccggcatcacggagcgcgagaagcgagagatcattgagaacgccgagaaggagaagagcccggagcagaatctgtttgagaagtacctggaacgccggggccgcagcaacttcctggccaagctgtacttggcacggcacgtcctgatcctgctgctcagcgtggtgcccatctcctacctatgcacatactacgccacccagaagcagaacgagttcacctgtgccctgggcgcctcacctgacgggccggtgggtagcgctgggcccacggtgcgcgtcagctgtaagctaccgtctgtgcagctgcagcggatcattgcaggtgtggacatcgtcttgctctgcttcatgaacctcatcatcctagtcaacctcatccacctcttcatcttccgcaagagcaacttcatcttcgacaaactaaacaaggtgggtatcaagacgcgccggcagtggcgccgctcgcagttctgcgacatcaacatcctggccatgttctgtaacgagaaccgcgaccacatcaagtcgctgaaccggctggacttcatcaccaacgagagcgacctcatgtacgacaatgtggtgcggcagcttctggctgccttggctcagtctaaccacgacaccacgcccactgtgcgggactctggcatccagaccgtggaccccagcatcaaccctgcggagcctgatggttctgctgagccacccgtggtcaagcggccccgtaagaaaatgaagtggatccccaccagcaacccgctgccacagcccttcaaggagcagctggccattatgcgcgtggaaaacagcaagactgagaagcccaagcccgtgcgcaggaagacagccacagacacacttattgccccgctgctggacgctggtgcccgtgctgcccaccactacaagggcagtggaggtgattcaggcccctcctctgccccgcctgctgcctctgagaaaaagcatacccgccacttctccttggacgtgcatccctatatcctaggtaccaagaaggccaagactgaggcggtaccccctgccctaccagcctcccgcagccaagaaggtggcttcctgtcccagacagaggaatgtgggctgggcctagctgcagcacccaccaaagagatgctccactccccgagaaggaaatcccgtaccccacagagcctgccctgccagggctcccatctgggggatcattccatgtctgctcaccccccgcagcccccgccgctgcttccctgtcaccaggcagtctgggcaaggctgaccctctcaccatcctgagccggaacgccactcaccccctgctccacatcagcacgctatcctcatcgccaccttcgaggagccgagaacagttgtgagtactgtggagttttgag

SEQ ID NO: 16 - protein, 607 aa, Mus musculus
MATALLAGEKLRELILPGSQDDKAGALAALLLQLKLELPFDRVVTIGTVLVPILLVTLVFTKNFAEEPIYCYTPHNFTRDQALYARGYCWTELRDALPGVDASLWPSLFEHKFLPYALLAFAAIMYVPALGWEFLASTRLTSELNFLLQEIDNCYHRAAEGRAPKIEKQIQSKGPGITEREKREIIENAEKEKSPEQNLFEKYLERRGRSNFLAKLYLARHVLILLLSVVPISYLCTYYATQKQNEFTCALGASPDGPVGSAGPTVRVSCKLPSVQLQRIIAGVDIVLLCFMNLIILVNLIHLFIFRKSNFIFDKLNKVGIKTRRQWRRSQFCDINILAMFCNENRDHIKSLNRLDFITNESDLMYDNVVRQLLAALAQSNHDTTPTVRDSGIQTVDPSINPAEPDGSAEPPVVKRPRKKMKWIPTSNPLPQPFKEQLAIMRVENSKTEKPKPVRRKTATDTLIAPLLDAGARAAHHYKGSGGDSGPSSAPPAASEKKHTRHFSLDVHPYILGTKKAKTEAVPPALPASRSQEGGFLSQTEECGLGLAAAPTKEMLHSPRRKSRTPQSLPCQGSHLGDHSMSAHPPQPPPLLPCHQAVWARLTLSPS

SEQ ID NO: 17 - DNA, 2476 bp, Mus musculus
ggctggtgctgggacactctccgctgaaaactagctgcctgaagctgtcactccactgtacctggatcgcttagggtagcattttcctctgtatccccccaagtccccattctcagcagcatcatgtcgctcgcacacactgctgcagagtacatgctctctgatgccctgctgcctgaccgcaggggctctcggctcaaaggactgcgcctggaactgcccctggataagatggtcaagttcattactgtgggctttcccttgctgctgatgtctctagctttcgcccaggagttctcatcagggtctccgatcagctgtttctctcccagcaacttcagtgtcaggcaggctgcatacgtggatagctcatgctgggactcgctggctcaccatacgcaggacaaggctggccagtacaaggtgaaatctctctggcctcacaaggctcttccctactctctactggctctggctgtggccatgtatctgccagtgttgctgtggcaatatgtagctgtgccctccctcagctctgacctgctgttcatcatcagtgagctggacaagtcttacaaccgttccatccgcctggtgcagcacatgttgcagatccggcagagcagctctgacccccatgtcttctgggatgagctagagaaggcccggaaggaacggtactttgaatttcccttggtagagcggtacctggaatgcaagcagcgctcccattggctagtggccacctacctcctgaggaacgccctcttactcctctttacctcagccacttacctgtaccttggccaattccatttagatgtcttctttcaagacgaatttaactgcttcatcaagacagggctgctacatgatgagacccatgtgccagagctgatcacatgcaggctgacctccctgtctgtattccagattgtgagtgtctccagtgcagcaatatacaccatattggtcccagtgataatatacaacctcacacggctatgtcggtgggacaaaggactcctatccatctatgagatgctcccagcttttgatctcctcagtagaaagatgctaggatgccccatcaatgacctcaatgtgattcttcttttcctccgagctaatatctctgagctcatctcttttagctggctgagtgtcttaagtgtgttgaaggatacaaccacccagaaacacaacatcgacacagtggttgactttatgacctttgtggctggcttagaaccctcaaaacctaaacatctcacccaacatacatatgatgaacacgcatagtttaaaaaaagaaacacagataagttttatagaaagtaactctccttcttcacaagctatgcagagtaaaagacagaaaaataataaggatgtccacgtaaaatggttaagagattggattcagctgaaatatcctgtatttttaagaagataaaaatcaacacatgaaaattcagtgtgaagtctgggagtcaagagtcaaagaactgaaaagttcagtttagaacattcagaggacagtagtgctaaaggaacaagctgaagcaaagtgcaattattgaactcagaaaaattctaaaggccatttatggagggaaattctcacaaggcccacccgctctagaagtcatgcttcccttcccacaatgggcagggtagaacaatttaataagagattaataaggatatgaacccctgaaactctttgatttggcctgcccatcttgctttgtggacattttcagccttgccaagcactctatagcctgctaatgagacaagaaataatgatggctttaacttgtaatctgcttttgggacataaggacagagacaaagccataagttgaatctttaggagtccaatacgctttttatgatataaactgaggattcttttccacgagggctcagattatggactatgaacaccttctaaaatcttaatgaagcctaggtgtattcctaatagtccaaagcatactacagccaaccagtgaaggtcaggaggtgaaaaggctgctgccaagcctgaatacctgatttcaatgcctgggacccaaatggtgaaaggatagaaccgattctccaagttaccttctgatctccatacacaagaaaagtaaacaaaggagtattaacatgcaagtcccatcgatccacttctatacattctccattcactgtttcctcctggcatctaattgtcacagcttagctctgattcaaaagacggtgctctatccccagtaccctcgtgactcttaataaatatttttaaactgtatattatgtatttcttgagagaatttttttatacaagtcatttagatccctcttcatataggtaatagaaaagtcaggttgcttctgacaaattcatgtcaattgccataaagcaagccaagagcttatagatgttcacagatgacattaataaaagtttcatgtcatt

SEQ ID NO: 18 - protein, 392 aa, Mus musculus
MSLAHTAAEYMLSDALLPDRRGSRLKGLRLELPLDKMVKFITVGFPLLLMSLAFAQEFSSGSPISCFSPSNFSVRQAAYVDSSCWDSLAHHTQDKAGQYKVKSLWPHKALPYSLLALAVAMYLPVLLWQYVAVPSLSSDLLFIISELDKSYNRSIRLVQHMLQIRQSSSDPHVFWDELEKARKERYFEFPLLERYLECKQRSHWLVATYLLRNALLLLFTSATYLYLGQFHLDVFFQDEFNCFIKTGLLHDETHVPELITCRLTSLSVFQIVSVSSAAIYTILVPVIIYNLTRLCRWDKGLLSIYEMLPAFDLLSRKMLGCPINDLNVILLFLRANISELISFSWLSVLSVLKDTTTQKHNIDTVVDFMTFVAGLEPSKPKHLTQHTYDEHA